SEQUENCE LISTING (4 sequences)

SEQ ID NO: 1 - DNA, 2300 bp, Homo sapiens
ggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggtaaaaaaaaaaaaaaaaaaaaaa

SEQ ID NO: 2 - DNA, 2267 bp, Homo sapiens
ggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggtaaaaaaaaaaaaaaaaaaaaaa

SEQ ID NO: 3 - DNA, 2221 bp, Homo sapiens
ggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgacccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggtaaaaaaaaaaaaaaaaaaaaaa

SEQ ID NO: 4 - DNA, 2189 bp, Homo sapiens
ggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaagggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggtaaaaaaaaaaaaaaaaaaaaaa

(19) United States
(12) Patent Application Publication
(10) Pub. No.: US 2018/0147296 A1
(43) Pub. Date: May 31, 2018

(54) CARBON MATERIAL DELIVERY SYSTEMS AND METHODS

(71) Applicant: The Research Foundation for The State University of New York, Albany, NY (US)

(72) Inventors: Balaji SITHARAMAN, Coram, NY (US); Sayan Mullick CHOWDHURY, Stony Brook, NY (US)

(73) Assignee: The Research Foundation for The State University of New York, Albany, NY (US)

(21) Appl. No.: 15/575,904

(22) PCT Filed: May 26, 2016
(86) PCT No.: PCT/US16/34229

§ 371 Date: Nov. 21, 2017

Related U.S. Application Data
(60) Provisional application No. 62/166,307, filed on May 26, 2015.
(60) Provisional application No. 62/244,219, filed on Oct. 21, 2015.

(51) Int. Cl. A61K 47/69; A61P 35/00; C12N 15/113; A61K 31/713; A61K 38/17; A61K 31/382; A61K 31/164; A61K 31/4245

(52) U.S. Cl. CPC A61K 47/6923; A61P 35/00; C12N 15/113; A61K 31/713; A61K 38/1709; C12N 2310/351; C12N 15/1137; A61K 31/164; A61K 31/4245; C12N 2310/14; C12N 2320/32; A61K 31/382

(57) ABSTRACT

The present disclosure is directed to carbon nanomaterials and methods for delivering a drug to a mammal's cell including administering the carbon nanomaterial to the mammal. The present disclosure is also directed to carbon nanomaterials and methods for delivering a nucleic acid to a mammal's cell including administering the carbon nanomaterial to the mammal.

### CROSS REFERENCE TO RELATED APPLICATIONS

The present application claims benefit of U.S. Provisional Application No. 62/166,307, filed May 26, 2015 and U.S. Provisional Application No. 62/244,219, filed Oct. 21, 2015, the disclosure of which is incorporated herein by reference in its entirety.

### BACKGROUND OF THE DISCLOSURE

Therapeutic gene delivery allows replacement of a mutated gene, production of therapeutic proteins (from delivered genes) inside target cells, delivery of inhibitory RNA (RNAi) for down-regulation of a mutated or over-expressed gene and in some cases for “suicide therapy” to guide the diseased cells towards apoptosis. Gene therapy for many diseases including cancer, hemophilia, sickle cell anemia, cystic fibrosis and Alzheimer's' disease are under various stages of investigation. Naked genetic material has a very short serum half life (about 1 min for RNA and about 10 min for DNA) due to rapid breakdown by nucleases resulting in their low bioavailability and entry into target cells.

Hence, gene delivery agents are being developed to increase the efficiency of gene delivery. For successful gene delivery into cells/tissues, the agent has to overcome three main barriers. Firstly, DNA or RNA, a negatively charged molecule, has to be stably loaded/conjugated to the delivery agent. Secondly, the agent has to be taken up by the cells, either through endocytosis or through macropinocytosis. Thirdly, the delivery agent has to enter the nucleus and deliver the DNA. The vectors used for gene delivery can be broadly classified as viral or non-viral agents. Viral agents (e.g., retroviruses, lentiviruses, adenoviruses) have the ability to enter cells, escape the endocytotic and macropinocytotic vesicles, and enter the nucleus to deliver the genetic material, leading to a relatively high (80-90%) transfection efficiency. However, viral agents are limited in terms to the size of genetic material they can carry (only small DNA/RNA can be packaged into viruses). They may also induce adverse immune responses and viral DNA/RNA may also integrate into the chromosomal DNA of the target cell resulting in deleterious mutations.

Widely used non-viral agents (liposomes, polycations), at optimal transfection concentrations, also exhibit limitations. Liposomal agents show low (about 6 times lower) transfection efficiency with large-sized DNA (about 52000 bp) compared to smaller DNA (about 2900 bp), and some liposomal agents have been shown to induce inflammatory reactions. Polycations that show relatively good transfection efficiencies and can condense large DNA are toxic. Further, a non viral vector's transfection efficiency depends on its ability to escape endosomes and enter the nucleus. Nuclear entry of genetic materials (even when condensed by non-viral vectors) is difficult because of the small size of nuclear pores (about 10 nm), and typically depends on cell division (when the nuclear membrane disintegrates). Thus, in general, non-viral vectors are more efficacious in adherent immortal or secondary cells which can undergo cell and sub-optimal with primary cells.

There is a desire for a versatile (that delivers relatively large sized DNA and RNAi) gene delivery system that is exhibits relatively low cytotoxicity and relatively high transfection efficiency (i.e. translocates into cells, escapes endosomes and enters the nucleus to deliver loaded genetic material).

Therefore, what is desired is the ability to deliver nucleic acids and drugs without the downfalls of each of the traditional methods. Embodiments of the present disclosure provide methods that address the above and other issues.

### SUMMARY OF THE DISCLOSURE

The present disclosure is directed to carbon nanomaterials and methods for delivering a drug to a mammal's cell including administering the carbon nanomaterial to the mammal. The present disclosure is also directed to carbon nanomaterials and methods for delivering a nucleic acid to a mammal's cell including administering the carbon nanomaterial to the mammal.

### BRIEF DESCRIPTION OF THE DRAWINGS

The present disclosure will be better understood by reference to the following drawings of which:

FIG. 1A is a representative atomic force microscope (AFM) image of oxidized graphene nanoribbons (O-GNR) illustrating two unzipped O-GNRs of about 500 nm in length; FIG. 1B is a representative low resolution transmission electron microscope (TEM) image of O-GNR illustrating several unzipped O-GNR; and FIG. 1C is an illustrative schematic illustrating the unzipping of multi walled tubes to form O-GNR, coating with PEG-DSPE to form O-GNR-PEG-DSPE and loading with lucanthone of form O-GNR-PEG-DSPE-Luc.

FIG. 2 is representative data graphically showing concentration of lucanthone loaded after measurement of lucanthone in the supernatants after loading, using differing concentration ratios of O-GNRs to lucanthone.

FIG. 3A-3H illustrate flow cytometry based analysis showing internalization of O-GNR-PEG-DSPE with GBM and CG-4 cell lines. FIGS. 3A and 3D include CMV/U251, FIGS. 3B and 3E include A1-5/CMV/U251 cells and FIGS. 3C and 3F include CG-4. FIGS. 3A-3C were left untreated as a control while FIGS. 3D-3F were incubated with PI-loaded O-GNR-PEG-DSPEs for 24 hours. After 24 hours, about 67% of CMV/U251 and about 60% of A1-5/U251 showed significant PI fluorescence, while about 29% of CG-4 showed significant fluorescence, as shown in FIG. 3I.

FIGS. 4A-4D are Representative TEM images illustrating: in FIG. 4A uptake of large aggregates of O-GNR-PEG-DSPE by CMV/U251 in large vesicular structures (indicated with dark arrow); FIG. 4B uptake of smaller O-GNR-PEG-DSPE aggregates into multiple vesicular structures in CMV/U251 (indicated with dark arrow); FIG. 4C MCF-7 does not show high uptake of O-GNR-PEG-DSPE particles (only small aggregates were observed, indicated with dark arrow); and FIG. 4D large aggregates did not get taken up my MCF-7 (indicated with dark arrows). Both cell lines were exposed to 40 μg/ml O GNR-PEG-DSPE for 3 hours. The nucleus in all cells is indicated with light arrows. FIG. 4E is a graphical representation of a PrestoBlue® assay measuring viability of MCF-7 and CMV/U251 in co-culture conditions after being treated for 24 hours with either O-GNR-PEG-DSPE, O-GNR-PEG-DSPE loaded with 80 μM Luc, 80 μM Luc, or lysis buffer. (*) indicates significant difference compared to untreated control (N=3). FIG. 4F is a graphical representation of a PrestoBlue® assay measuring viability of CG-4 and CMV/U251 in co-culture conditions after being treated for 24 hours with either O-GNR-PEG-DSPE, O-GNRPEG-DSPE loaded with 80 μM Luc, 80 μM Luc, or lysis buffer. (*) indicate significant difference compared to untreated control (N=3).

FIGS. 5A-5D are graphical representations of PrestoBlue® assays measuring viability of 5×10³ CMV/U251 in FIG. 5A, A1-5 U251 in FIG. 5B, CG-4 in FIG. 5C and MCF-7 in FIG. 5D, after being treated for 24 hours with either P-GNR-PEG-DSPE, O-GNR-PEG-DSPE loaded with 80 μM Luc, 80M Luc or lysis buffer. (*) indicates significant difference compared to lysis control, (f) indicates significant difference compared to 8 μM Luc, and (‡) indicates significant difference compared to O-GNR-PEG-DSPE loaded with 80 μM Luc.

FIG. 6 illustrates flow cytometry based analysis of cell death in control CMV/U251 cells after 2 hours of treatment with different weights of O-GNR-PEG-DSPE used to load the different concentrations of lucanthone (0-80 μM) using Annexin V/PI staining.

FIG. 7 illustrates flow cytometry based analysis of cell death in control CMV/U251 and APE-1 overexpressor A1-5/CMV/U251 treated for 4 hours with different concentrations of Luc (5-80 μM) either alone or loaded onto O-GNR-PEG-DSPE using Annexin V/PI staining.

FIG. 8 graphically illustrates the viability of 5×10³ HeLa cells exposed to (A) O-GNR-PEGDSPE at 40 μg/ml (GO), (B) 3.1 μM lucanthone loaded O-GNR-PEG-DSPE (C) Free lucanthone at 1.55 μM (D) Free Lucanthone at 3.1 μM for 24 hours.** and * represents significant difference (p value <0.05) from unexposed control cells and 3.1 μM free lucanthone respectively, N=6.

FIG. 9A is a low resolution TEM image of O-GNR's produced from unzipping of multi-walled carbon nanotubes (MWCNT). FIG. 9B is a graphical representation of a comparison of toxicity of O-GNR, Fugene 6 and Polythyleneimine in MRC5 fibroblast cells at maximum concentration used for transfection. FIG. 9C is an illustrative schematic showing mechanism of loading of dsDNA onto O-GNR suspension in phosphate buffered saline. FIG. 9D is a graphical representation of loading efficiency of EGFP plasmid onto O-GNR (1.2 mg/mL) in phosphate buffered saline. FIG. 9E is a graphical representation of loading efficiency of siRNA against GAPDH onto O-GNR (1.2 mg/mL) in phosphate buffered saline.

FIGS. 10A-10C are representative confocal microscopy images of HeLa cells treated with various concentrations of EGFP plasmid loaded O-GNRs. Images represent nuclear staining with Hoechst stain and EGFP expression respectively in cells treated with FIG. 10A 20 μg/ml, FIG. 10B 40 μg/ml and FIG. 10C 60 μg/ml GNR. FIG. 10D is a graphical representation of the quantification of EGFP fluorescence in HeLa cells treated with various concentrations (20-60 μg/mL) of EGFP loaded O-GNR's. FIG. 10E is a graphical representation of the quantification of GAPDH activity in HeLa cells treated with various concentrations (10-60 μg/mL) of siRNA (against GAPDH) loaded O-GNR's.

FIGS. 11A-11C are representative confocal microscopy images of HUVEC cells treated with various concentrations of EGFP plasmid loaded O-GNRs. These figures represent nuclear staining with Hoechst stain and EGFP expression respectively in cells treated with (FIG. 11A) 20 μg/mL GNR, (FIG. 11B) 40 μg/mL GNR and (FIG. 11C) 60 μg/mL GNR. FIG. 11D is a graphical representation of the quantification of EGFP fluorescence in HUVEC cells treated with various concentrations (20-60 μg/mL) of EGFP loaded O-GNRs. FIG. 11E is a graphical representation of the quantification of GAPDH activity in HUVEC cells treated with various concentrations (10-60 μg/mL) of siRNA (against GAPDH) loaded O-GNRs.

FIGS. 12A-C are representative brightfield, merge and fluorescence images of HeLa cells treated with fluorescein isothiocyanate (FITC) FITC-PEG-DSPE loaded O-GNR particles. The dark arrows indicate cells and the light arrows indicate nanoparticles in the figures. FIGS. 12D-12I are representative TEM of HeLa and HUVEC cells showing: in FIGS. 12D and 12I invagination of cell membrane and formation of vesicles at the surface of the membrane in contact with O-GNR; FIGS. 12E and 12H a vesicle containing large O-GNR aggregates; FIG. 12F a HeLa cell showing membrane extensions taking up O-GNR aggregates; and FIG. 12I a HUVEC cell showing uptake of O-GNR into large number of vesicular structures throughout the cytoplasm. In FIGS. 12 D-12I the lightest arrows indicate cell membrane, darkest arrows indicate nanoparticles, and dark arrows indicate formation of vesicles and green arrows indicate the nuclear membrane.

FIGS. 13A-13F are representative TEM images of: FIG. 13A) a HeLa cell showing O-GNR aggregates escaping a vesicle; FIG. 13B a HeLa cell showing an escaped O-GNR aggregate outside the nuclear membrane; FIG. 13C a HeLa cell showing two O-GNR aggregates released from lysed vesicles; FIG. 13D a HeLa cell showing a large O-GNR aggregate released from a lysed vesicle; FIGS. 13E-13F HUVEC cells showing escaped O-GNR aggregates outside the nuclear membrane. In FIGS. 13A-13F the darkest arrows indicate O-GNR particles inside vesicles, the lightest arrows indicate nuclear membrane, dark arrows indicate escaped or escaping nanoparticles and light arrows indicate the vesicles.

FIGS. 14 A-D illustrate flow cytometry based analysis showing PI fluorescence in 10⁴ HeLa cells either left untreated (FIG. 14A) or incubated with PI-loaded O-GNRs for 2 hours (FIG. 14B), 12 hours (FIG. 14C) or 24 hours (FIG. 14D). FIG. 14E is a graphical representation of the percentage of cells showing PI fluorescence at the three time points tested calculated from the flow cytometry data.

FIGS. 15A-15F are representative TEM images of HeLa cells treated with 20 μg/ml O-GNR for 12 hours (FIG. 15A) O-GNR aggregates inducing formation of nuclear invagination. The figures also show presence of O-GNR's in the nucleus (FIG. 15B) O-GNR aggregates entering the cell and nucleus (FIG. 15C) O-GNR aggregates inside the nucleus. FIGS. 15D-15F are brightfield (FIG. 15D), Hoechst fluorescence (FIG. 15E) and a merger of Hoechst and FITC fluorescence images (FIG. 15F) of HeLa cells treated with FITC-PEG-DSPE loaded O-GNR's for 12 hours. FIGS. 15G-15I are representative TEM images of HUVEC cells treated with 20 g/ml O-GNR for 12 hours showing: (FIG. 15G) O-GNR aggregates inducing formation of nuclear invagination; (FIG. 15H) creation of a gap in the nuclear membrane with O-GNR's also being seen near the gap; and (FIG. 1) the presence of O-GNR inside the nucleus. In all TEM images the darkest arrows indicate O-GNR particles, the light arrows indicate nuclear membrane, the lightest arrows indicate invaginations of the nuclear membrane and the dark arrows indicate darkening of the nucleus.

FIG. 16 is a graphical representation of cell counts using PrestoBlue® Assays before and after 12 hour treatment of HeLa and HUVEC cells with FITC-O-GNRs.

FIG. 17 is an illustrative schematic representation of the probable mechanisms of nuclear transfection by O-GNRs.

FIGS. 18A-18H are Confocal Microscopy images.

FIGS. 19A-19D are graphical illustrations of viability of HeLa cells.

FIGS. 20A and 20B are graphical illustrations of apoptosis of HeLa cells.

FIGS. 21A and 21B are Atomic Force Microscopy images of 21A) O-GNRs and 21B) GNPs.

FIGS. 22A-22C are mass spectrometry readouts showing the relative abundance of C6 over time for 22A) the C6 ceramide stock solution 22B) C6 ceramide loaded onto O-GNRs 22C) and C6 ceramide loaded onto GNPs.

FIGS. 23A and 23B are graphical representations of the viability of HeLa cells assessed by PrestoBlue after 24 hrs of incubation with 23A) C6-loaded O-GNRs or 23B) C6-loaded GNPs. O-GNRs and GNPs not loaded with C6 served as controls. * indicates significant difference from “Untreated.” t indicates significant difference from “100 μg/mL O-GNRs/GNPs only.”

FIGS. 24A and 24B are graphical representations of the viability of HeLa cells assessed by PrestoBlue after 24 hrs of incubation with 24A) C6-loaded O-GNRs resuspended in PEG-DSPE or 24B) C6-loaded GNPs resuspended in PEG-DSPE. O-GNRs and GNPs not loaded with C6 served as controls. * indicates significant difference from “Untreated.” t indicates significant difference from “100 μg/mL O-GNRs/GNPs only.”

FIG. 25. is a graphical representation of the viability of HeLa cells assessed by PrestoBlue after 24 hrs of incubation with C₆-loaded O-GNRs or C₆ alone. * indicates significant difference from “C6 only” at the respective concentration.

FIGS. 26A-26D are images of Hoechst staining for apoptosis in HeLa cells after 24 hour exposure to C6-loaded O-GNRs (26A, 26B) or O-GNRs alone (26C, 26D). 26A) Cells exposed to C6-loaded O-GNRs display bright nuclei after 24 hours, indicating apoptosis. 26B) A single cell nucleus displaying fragmentation characteristic of apoptosis after treatment with C6-loaded O-GNRs. 26C) Cells exposed to O-GNRs only do not demonstrate significant levels of apoptosis after 24 hours. 26D) A closer image of cells treated with only O-GNRs shows that these cells come in contact with high numbers of O-GNRs (dark spots, indicated by arrows).

FIGS. 27A-27F are time-lapsed confocal microscopy images of HeLa cells incubated with NBD ceramide loaded O-GNRs 27A) after initial additional of O-GNRs (t=0 minutes), 27B) after 12 minutes, 27C) after 24 minutes, 27D) after 36 minutes, 27E) after 48 minutes, and 27F) after 60 minutes. There is no fluorescence from ceramide present initially (27A) and some fluorescence present after 24 minutes have past (27B and 27C). There is a large increase in fluorescence between 24 minutes and 36 minutes, possibly due to uptake of NDB-ceramide loaded O-GNRs (27D). This fluorescence does not diminish over time, indicating a non-transient effect (27E and 27F).

FIGS. 28A and 28B are live Cell Confocal Microscopy images of HeLa cells (28A) prior to addition of NBD-ceramide and (28B) after 7 minutes of exposure to NBD ceramide.

FIGS. 29A-29D are transmission electron microscopy images of HeLa cells incubated with C6 ceramide-loaded O-GNRs after one hour. 29A) After one hour, there are many O-GNRs present in the cells contained in vacuoles. 29B) A zoomed-in perspective shows O-GNRs of different sizes and shapes in these vacuoles. 29C) Uptake of O-GNRs into HeLa cells shows the same macropinocytotic-like features found in previous studies (indicated by arrows). 29D) A closer view reveals the blebbing of the cell membrane around the O-GNRs characteristic of the macropinocytotic-like process previously found, indicating that loading of ceramide onto O-GNRs does not significantly alter the process by which these particles enter cells.

### DETAILED DESCRIPTION OF THE DISCLOSURE

As used herein, the term graphene refers to one or more atomic layers of graphite, e.g., with a single graphene layer or being extendible up to n-layers of graphite (e.g., where n can be as high as about 30). Graphene can be a single layer of covalently bonded carbon atoms (Generally, sp² bonds). The graphene can have various shapes including three-dimensional shapes such as a sheet or layer shape, a ribbon shape, a spherical shape, a cylindrical shape, and a polyhedral shape.

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein, the term carbon nanotube refers to an elongated hollow structure having a cross section (e.g. angular fibers having edges) or a diameter (e.g. rounded) less than about 1 micron.

As used herein, the term graphene oxide nanoribbon refers to, for example, single- or multiple layers of graphene (typically less than 10 carbon layers thick) that have an aspect ratio of greater than about 5, based on their length and their width.

As used herein the term graphene oxide nano-onion shall mean hollow, porous, multi-wall carbon nanospheres or polyhedral structures with a narrow size distribution and an average particle size of approximately 80 nm and an average aspect ratio close to 7:5. Such structures are also referred to as carbon Q-dots or Q-graphene and can be any suitable sp² hybridized carbon nanostructure.

The disclosure includes a method of delivering a drug to a mammal's cell and a carbon nanomaterial. The mammal can be any suitable mammal, including, without limitation, humans, canines, horses, felines, and livestock. The cell to which the drug is delivered can be one or more targeted cells, including but not limited to glioblastoma cells.

The carbon nanomaterial can be any suitable carbon nanomaterial that is capable of and configured to enter the cell of the animal. This carbon nanomaterial includes but is not limited to multi-walled carbon nanotubes (MWCNT), nanotubes, graphenes, fullerenes, graphene oxide nanoplatelets, graphene oxide nanoribbon, graphene oxide nano-ions and combinations thereof.

The method for delivering a drug to a mammal's cell includes the step of administering a carbon nanomaterial to the mammal. This administration can occur in any suitable way that is capable of delivering the drug to the selected cell, such as intravenously and injection with a needle or the like.

Optionally, carbon nanomaterial loaded with drug can be coated or non-covalently functionalized with a polymer prior to administration, with that polymer being any suitable polymer including, but not limited to, 2-distearoyl-sn-glycero-3-phosphoethanolamine-N-[amino (polyethylene glycol)), dextran and combinations thereof. As used herein, the term functionalized or functionalization refers to the addition of a material, other than the drug or nucleic acid, to the carbon nanomaterial that forms a covalent bond or non-covalent bond with the carbon nanomaterial and modifies some feature of the carbon nanomaterial. Options for materials that can be added to the carbon nanomaterial-drug/nucleic acid complex so that they are functionalized carbon nanomaterial-drug/nucleic acid complexes, include but are not limited to Dextran, PEG, etc. Although optional, functionalization can increase dispersability of the carbon nanomaterial—drug/nucleic acid complexes in a diluent and can also protect and mitigate the degradation of the nuclei acid and carbon nanomaterials by enzymes over time.

The drug that is delivered by the carbon nanomaterial can be any suitable drug that can affect a cell by entering the cell. This drug can be any suitable drug, including but not limited to one or more selected from the group consisting of lucanthone, doxorubicin, paclitaxel, camptothecin, tamoxifen, ceramide and combinations thereof.

The drug that is delivered with the carbon nanomaterial can be for any therapeutic purpose, such as treatment of diseases.

The disclosure includes a method of delivering a nucleic acid, including a suitable protein, to a mammal's cell and a carbon nanomaterial. The mammal can be any suitable mammal, including, without limitation, humans, canines, horses, felines, and livestock. The cell to which the drug is delivered can be one or more targeted cells, including but not limited to cervical cancer cells and brain cells.

The carbon nanomaterial can be any suitable carbon nanomaterial that is capable of and configured to enter the cell of the animal. This carbon nanomaterial includes but is not limited to multi-walled carbon nanotubes (MWCNT), nanotubes, graphenes, fullerenes, graphene oxide nanoplatelets, graphene oxide nanoribbon, graphene oxide nano-ions and combinations thereof.

One method for delivering a nucleic acid, or protein, to a mammal's cell includes the step of administering a carbon nanomaterial to the mammal. Another method for delivering a nucleic acid to a cell includes the step of administering a carbon nanomaterial to a cell culture, or other suitable in vitro cell population. This administration can occur in any suitable way that is capable of delivering the nucleic acid to the selected cell, such as intravenously, injection with a needle or the like and using a suitable delivery device such as a pipette. The intravenous administration can transport the carbon nanomaterial and the nucleic acid or protein from the injection site to brain cells of the mammal, through the blood brain barrier due to the small size of the carbon nanomaterial.

Optionally, the carbon nanomaterial loaded with nucleic acid can be coated with a polymer prior to administration, with that polymer being any suitable polymer including, but not limited to, 2-distearoyl-sn-glycero-3-phosphoethanolamine-N-[amino (polyethylene glycol)), dextran and combinations thereof.

The nucleic acid that is delivered by the carbon nanomaterial can be any suitable nucleic acid that can affect a cell by entering the cell. This nucleic acid can be any suitable nucleic acid, including but not limited to a plasmid, DNA molecule and siRNA molecule. The protein that can be delivered by the carbon nanomaterial can be any suitable protein that can affect a cell by entering the cell. This protein can be any suitable protein, including but not limited to, Myelin Basic Protein (MBP), including portions and fragments thereof.

The nucleic acid or protein that is delivered with the carbon nanomaterial can be used for any therapeutic purpose or research purpose, such as treatment of a disease, disorder-including neurological disorders, replacement of a mutated gene, production of therapeutic proteins, delivery of inhibitory RNA for down-regulation of a mutated or over-expressed gene or cause apoptosis in a cell.

Additionally, recently there has been a growing appreciation for the active roles certain lipids take in cellular processes. Of particular interest is the group of sphingolipids known as ceramides. These lipids are composed of sphingosine linked to a fatty acid by an amide bond, and different biological effects on cells depending on the length of the fatty acid chain have been reported. Ceramide C₂₄, for example, generally has roles in cell proliferation and survival, while ceramide C₁₆ has anti-proliferative effects and is associated with apoptosis_ENREF_7_ENREF_8.

Due to graphenes sp² hybridized orbital structure, pristine graphene is hydrophobic. However, it can be oxidized to a more water-dispersible form while still retaining hydrophobic sp² moieties, giving rise to oxidized graphene nanoparticles.

However, these hydrophobic portions of graphene are also capable of binding non-aromatic hydrophobic molecules; for example, it is possible to increase the dispersibility of graphene oxide in aqueous solution using surfactants such as 1,2-distearoyl-sn-glycero-3-phosphoethanolamine-N-[amino(polyethylene glycol)] (DSPE-PEG). Also, graphene oxide nanoparticles can bind ceramide given a loading method that facilitates hydrophobic interactions between the two while also being compatible with ceramide's hydrophobicity.

As discussed in more detail in Example 4 below, a method for loading the short-chain C₆ ceramide onto graphene nanoparticles is disclosed. By shifting the environment of a ceramide-graphene nanoparticle mixture from hydrophobic to aqueous, relatively high levels of ceramide loading can be realized. Furthermore, these ceramide-loaded nanoparticles are able to enter HeLa cells and induce toxicity.

As discussed in more detail in Example 5 below, graphene nanoparticles can be bound to nucleic acids, including proteins and Myelin Basic Protein for administration to a mammal and delivery to the mammal's brain. This administration to the mammal's brain can be for treatment of neurological disorders.

As used herein, the term “treatment” or “treating” refers to any means of control of the conditions, including prevention, cure and relief of the conditions and arrestation or relief of development of the condition.

As used herein, the term “neurological disorder” refers to strokes, traumatic brain injury, cerebral palsy, dystonias, hydrocephalus, toxicity, inflammation, muscular dystrophies, motor neuron diseases, inflammatory myopathies, neuromuscular junction disorders, peripheral nerve disorders, as well as neurodegenerative disorders such as, multiple sclerosis, Parkinson's disease and other neurological conditions resulting in a reduction of motor function. Examples of motor neuron diseases include, but are not limited to, adult spinal muscular atrophy, amyotrophic lateral sclerosis or Lou Gehrig's Disease, infantile progressive spinal muscular atrophy or SMA Type 1 or Werdnig-Hoffman, intermediate spinal muscular atrophy or SMA Type 2, juvenile spinal muscular atrophy or SMA Type 3 or Kugelberg-Welander, spinal bulbar muscular atrophy (SBMA) or Kennedy's Disease, or X-linked SBMA. Examples of neuromuscular junction diseases include, but are not limited to, myasthenia gravis, Lambert-Eaton Syndrome, and congenital myasthenic syndrome. Examples of peripheral nerve disorders include, but are not limited to, Charcot-Marie-Tooth Disease or peroneal muscular atrophy, Dejerine-Sottas Disease, and Friedreich's Ataxia. Other myopathies include myotonia congenita or Thomsen's and Becker's Disease, paramyotonia congenita, central core disease, periodic paralysis (PP) hypokalemic and hyperkalemic, endocrine myopathies, and mitochondrial myopathies.

The term “stroke” refers to the multitude of subcategories of cebrovascular diseases including thrombotic or embolic infarction as well as intracerebral hemorrhage from a vascular or post operative nature.

The methods, apparatus and compositions of the present disclosure will be better understood by reference to the following Examples, which are provided as exemplary of the disclosure and not by way of limitation.

### Example 1

One Example of a drug delivery method and system is discussed below. Each of the delivery methods and systems discussed herein can be delivered to a subject in any suitable way, including intravenously and through targeted injections.

### Reagents

Cell line U251 and reagents used for measuring endonuclease activity were provided. CG-4, a rat glial progenitor cell line was maintained at 4° C. under hygroscopic conditions, and dissolved in 1.2 mg/mL 1,2-distearoyl-sn-glycero-3-phosphoethanolamine-

N-[amino(polyethylene glycol (PEG-DSPE) (in double distilled water) just prior to reactions. Plasmids consisting of full length Apurinic endonuclease-1 (APE-1) in pCMV10 were obtained. Multi-walled carbon nanotubes (MWCNTs) and propidium iodide (PI) were obtained from Sigma Aldrich. All cell culture components were obtained from GIBCO. Annexin V/PI staining kits were obtained from Trevigen

### Cell Culture

U251 transfected with either the blank plasmid pCMV10 (CMV/U251) or full length APE-1 in pCMV10 (A1-5/CMV/U251) were grown in Dulbecco's Modified Eagle Medium (DMEM) supplemented with 10% fetal bovine serum and 800 μg/mL of G418. CG-4 were grown in 70% of DMEM F12 containing 1× penicillin streptomycin (100 μg/mL Streptomycin+100 U of penicillin) (PS) with 1×N2 supplement (containing 1 mM Transferrin, 0.06 mM Insulin, 0.002 mM progesterone, 10 mM putresceine and 0.003 mM selenite) and 30% of B104 conditioned medium. MCF-7 were grown at 37° C. in a humidified atmosphere of 5% CO2 in RPMI medium supplemented with 10% fetal bovine serum and 1×PS.

### O-GNR Synthesis

Oxidized graphene nanoribbons (O-GNRs) were synthesized from MWCNTs (Sigma-Aldrich, Length=5-9 μm) using the oxidative longitudinal unzipping method. MWCNTs (150 mg) were suspended in 30 mL concentrated (96%) H₂SO₄. After 4 hours, 4.75 mM KMnO₄ was added slowly and stirred for one hour followed by further stirring for another hour at 55-70° C. in an oil bath. This solution was poured on ice (400 mL) containing 5 mL 30% H₂O₂ and the ice-H₂O₂ slurry was allowed to melt. The solution obtained was centrifuged at 3000 rpm for 30 minutes, after which the supernatant was discarded. The pellet obtained was then washed with 36% HCl. Ethanol and ether washes were used for flocculation and the final product (O-GNR) was obtained as pellet after centrifugation (30 minutes, 3000 rpm).

This product was dried overnight in a vacuum oven at 60° C. O-GNRs were characterized using atomic force microscopy (AFM) and transmission electron microscopy (TEM). AFM images were obtained using a Nano Surf Easy Scan 2 AFM (NanoScience Instruments Inc, Phoenix, Ariz.) operating in tapping mode using a V-shaped cantilever and TEM images were obtained using a Tecnai Bio Twin G transmission electron microscope (FEI, Hillsboro, Oreg.), at 80 kV.

### Luc Loading on O-GNR-PEG-DSPE

Powdered O-GNRs were dispersed in a solution of 1.2 mg/mL 1, 2-distearoyl-sn-glycero-3-phosphoethanolamine-N-[amino (polyethylene glycol)) (PEG-DSPE), at a concentration of 1 mg/mL. This dispersion was bath sonicated for 30 minutes to produce PEG-DSPE coated O-GNR (O-GNR-PEG-DSPE). The PEG-DSPE coats the O-GNR through non-covalent interaction, the DSPE is hydrophobic and has a van der Wall interaction with the hydrophobic graphene. 1 mg/mL solution of Lucanthone (Luc) in 1.2 mg/mL PEG-DSPE served as a Luc stock solution. 200 μL of the O-GNR-PEG-DSPE solution and 400 μL of the Luc solution were combined in a 20 mL glass vial, and the total volume was made up to 1 mL using a stock solution of 1.2 mg/mL PEG-DSPE and stirred at 4° C. for 24 hours. The Luc is “loaded” onto the O-GNR-PEG-DSPE through a non-covalent interaction, also referred to as pi-pi interaction. After this loading period, unincorporated Luc was separated out from the loaded O-GNRs by centrifugation at 13,000 RPM for 1 hour. In order to calculate the loading efficiency, the absorbance of the supernatant was measured at 328 nm using an Evolution 300 UV-vis Spectrophotometer (Thermo Fisher Scientific, Waltham, Mass.) and compared to a standard curve. The loading efficiency was calculated by subtracting the unloaded Luc from the total available Luc. Loaded O-GNR-PEG-DSPEs were left in pellet form until used.

### Uptake of O-GNR-PEG-DSPE in the U251 and CG-4 Cell Lines (Using Flow Cytometry)

O-GNR-PEG-DSPEs were loaded with PI and purified using the same method used for Luc loading. Cytomegalovirus (CMV) CMV/U251 cell line and A1-5/CMV/U251 were grown in 10 cm dishes at 37° C. and 5% CO₂ in DMEM. Cells were either incubated with PI-loaded O-GNRPEG-DSPEs at a concentration of 40 μg per mL of media, or left untreated. After 24 h, cells were trypsinized, resuspended in FACS buffer (1×PBS containing 20% fetal bovine serum) and placed on ice. Flow cytometry was performed immediately after all samples were prepared using a FACS Calibur Cell Sorter (BD Biosciences, San Jose, Calif.).

### Transmission Electron Microscopy

Six well plates with surfaces covered with ACLAR® film (Electron Microscopy Sciences, Hatford, Pa.) were plated with CMV/U251 and MCF-7 cells at a density of 5×10⁵ cells per plate, and exposed to O-GNR-PEG-DSPE for 3 hours. At the end of 3 hours, cells were fixed with 2.5% electron microscopy grade glutaraldehyde (ElectronMicroscopy Sciences, Hatford, Pa.) in 1×PBS. After fixation, the films containing fixed cells were placed in 2% osmium tetroxide in 1×PBS, dehydrated through graded ethanol washes, and embedded in durcupan resin (Sigma-Aldrich, St. Louis, USA). Areas with high cell densities were blocked, cut into 80 nm ultra-thin sections using an Ultracut E microtome (Reichert-Jung, Cambridge, UK), and placed on formvar-coated copper grids. The sections were then viewed with a Tecnai BioTwin G transmission electron microscope (FEI, Hillsboro, Oreg.), at 80 kV. Digital images were acquired using an XR-60 CCD digital camera system (AMT, Woburn, Mass.).

### PrestoBlue® Assay

In Separate Cultures

Luc alone (80 μM), 80 μM Luc loaded O-GNR-PEG-DSPE and O-GNR-PEG-DSPE (same weight as used for Luc loading) were incubated with CMV/U251, A1-5/CMV/U251, MCF-7 and CG-4 cells (5×103 cells per well in 96 well plates) for 24 hours. Following the incubation period the media was removed and the cells were washed twice with 1×PBS. 100 μL of fresh media was added to each well, as well as 10 μL of PrestoBlue® to each well, and incubated for 4 hours. Fluorescence was measured by Cytofluor fluorescence multiwell plate reader (Infinite M200, Tecan Group Ltd, NC, USA) with excitation at 530 nm, and emission at 580 nm.

The cell viability in terms of % of unexposed cells is expressed as the percentage of (Fₜₑₛₜ−F_{blank})/(F_{control}−F_{blank}), where Fₜₑₛₜ is the fluorescence of the cells exposed to nanoribbon-drug sample, F_{control} is the fluorescence of the untreated sample and F_{blank} is the fluorescence of the wells without any cells. The PrestoBlue® reading for all lysed cells was also taken for comparison.

MCF-7-CMV/U251 and CG-4-CMV/Co-Culture

MCF-7, CG-4 and CMV/U251 were seeded separately on 12 mm round glass coverslips (Fisherbrand, Fisher Scientific, PA) in 100 mm plates at a density of 2.2×10⁶ cells and allowed to grow overnight. For the two separate co-culture condition experiments, one coverslip from each cell line was placed in 6 well plates, for a total of two coverslips per well. These cells were then incubated with Luc alone (80 μM), 80 μM Luc loaded O-GNR-PEG-DSPE or O-GNR-PEG-DSPE for 24 h. After the incubation period, the media was removed and the cells were washed twice with 1×PBS. Each coverslip was then transferred to its own well in 6 well plates containing 3 mL of media and 300 μL of PrestoBlue® reagent. After incubating for 4 h, the fluorescence intensity was recorded using the procedure described above.

### Annexin V/PI Staining

Luc alone and Luc-loaded O-GNR-PEG-DSPEs at different concentrations (5-80 μM) were incubated with CMV/U251, A1-5/CMV/U251 and CG-4 (5×10⁵ cells/3 mL in 6-well plates) for 4 hours. Unexposed cells were used as control. Unloaded O-GNR-PEG-DSPEs in amounts equivalent to the loaded nanoparticles were also exposed to all the cell types as delivery agent control. The viability of the cells was analyzed by flow cytometry after Annexin V/PI staining using FACS Calibur Cell Sorter (BD Biosciences, San Jose, Calif.).

### Statistics

PrestoBlue® data in this Example is presented as mean±standard deviation (n=6). Student t test was used to analyze the differences among groups. One-way ANOVA followed by Tukey Kramer post hoc analysis was used for multiple comparisons between groups. All statistical analyses were performed using a 95% confidence interval (P b 0.05).

### AFM and TEM Analysis of O-GNRs

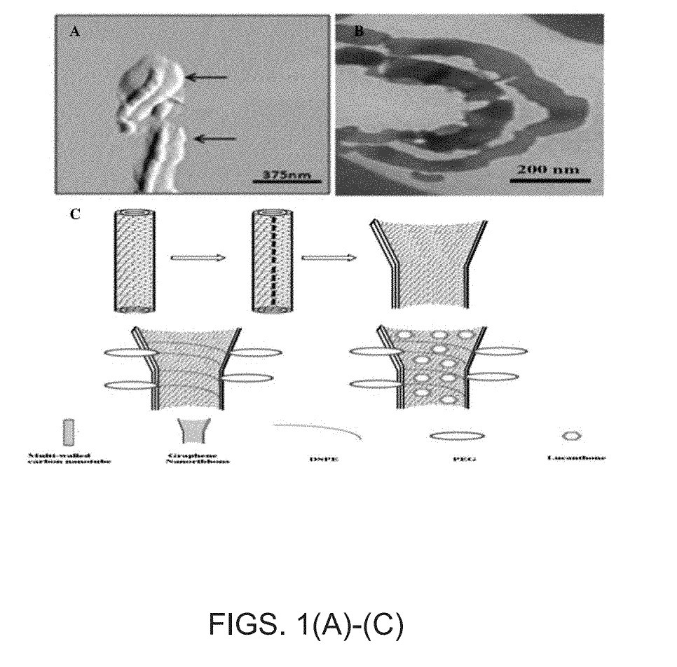

FIGS. 1A and 1B are representative AFM and TEM images of the O-GNRs. The O-GNRs are relatively thin, elongated strips of graphene that possess straight edges with an average breadth of about 100 to about 300 nm and a length of about 500 to about 2500 nm as determined from six different AFM images. FIG. 1A shows two unzipped nanoribbons with lengths of about 500 nm each. FIG. 1B shows multiple unzipped nanoribbons with lengths between 1000 and 2500 nm.

### Luc Loading on O-GNRs

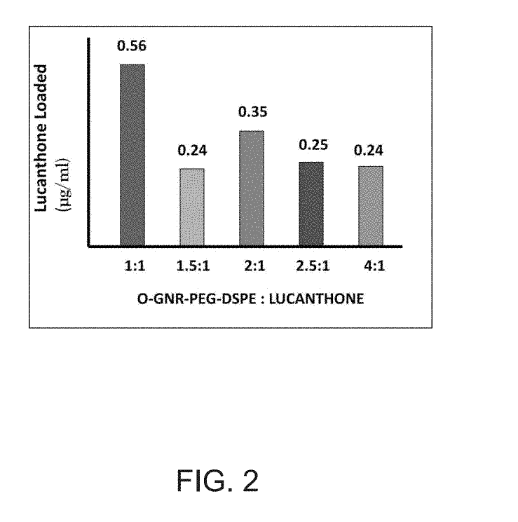

Using a standard curve of Luc, and employing a previously determined ratio of 1:1.5 of Luc:O-GNR, it was determined that O-GNR-PEG-DSPE could load 310 μM of Luc per mg, which is graphically shown in FIG. 2. A schematic of the loading process is shown in FIG. 1C.

### Uptake of O-GNR-PEG-DSPEs in U251 Cells

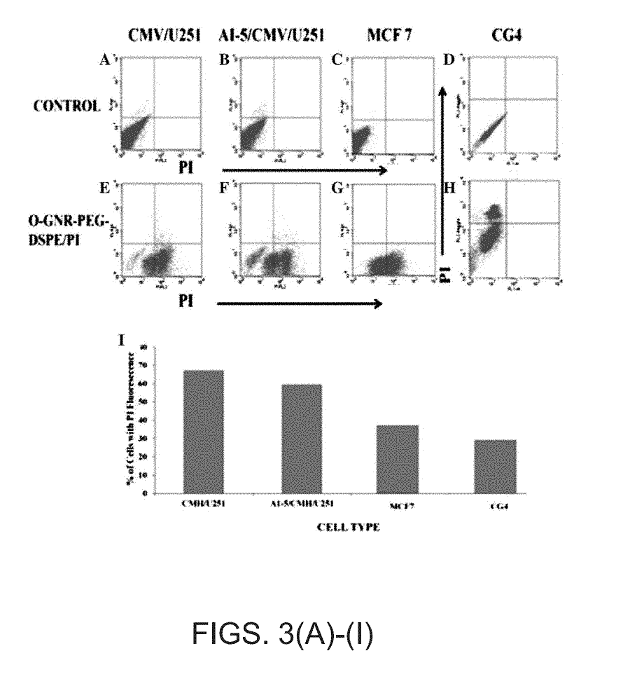

PI is a DNA intercalating fluorescent dye that it is typically excluded from live cells. However, when conjugated to a delivery agent such as O-GNR-PEG-DSPE it can enter live, intact cells. FIGS. 3A-3I illustrate flow cytometry analysis of U251 and CG-4 incubated with PI loaded O-GNR-PEG-DSPE revealed that about 60% of A1-5/CMV/U251 showed uptake of PI-loaded O-GNR-PEG-DSPEs, while about 67% of CMV/U251 showed uptake. About 38% of MCF and about 29% of CG-4 showed uptake of the PI loaded O-GNR-PEG-DSPE.

### TEM

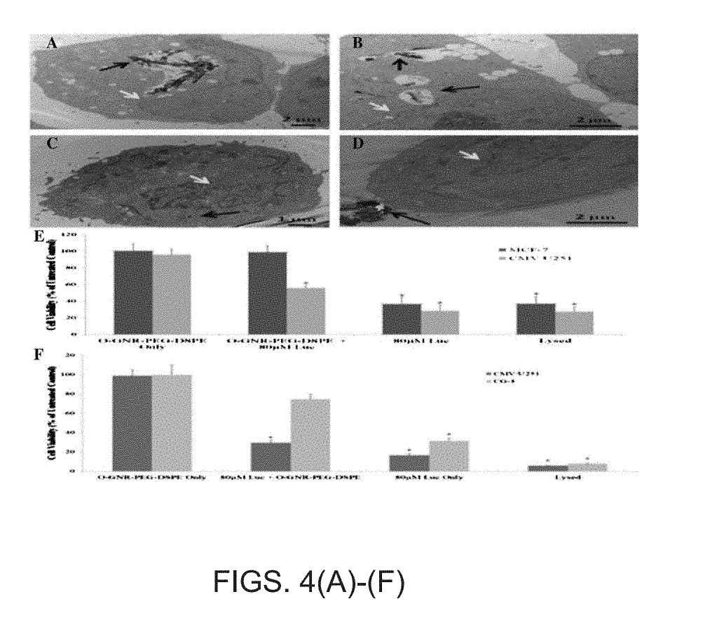

FIGS. 4A and 4B show that CMV/U251 could take up large aggregates of O-GNR-PEG-DSPE into large vesicular structures (black arrows) that localized near the nucleus (white arrows). FIG. 4C shows that MCF did not show uptake of large aggregates although few small aggregates were observed (black arrow). FIG. 4D shows that large aggregates of O-GNRPEG-DSPE on the surface of MCF-7 (black arrow) did not get taken up by MCF-7.

### MCF-7-CMV/U251 and CG-4-CMV/U251 Co-Cultures

FIG. 4E shows that MCF-7 and CMV/U251 treated with 80 μM Luc exhibit a viability of about 36% and about 28% respectively when compared to untreated controls. CMV/U251 still exhibited significant decreased viability when exposed to Luc-loaded O-GNR-PEG-DSPE, with only about 56% viability compared to untreated control. However, MCF-7 exposed to Luc-loaded O-GNR-PEG-DSPE do not exhibit decreased viability compared to untreated controls. O-GNR-PEG-DSPE alone was not toxic to either cell line. This trend held true for the CG-4 and CMV/U251 co-culture also (FIG. 4F). CG-4 and CMV/U251 treated with 80 μM Luc exhibit a viability of about 30% and about 15% respectively when compared to untreated controls. CMV/U251 exhibited significant decreased viability when exposed to Luc-loaded O-GNR-PEG-DSPE, with only about 29% viability compared to untreated control. However, CG-4 exposed to Luc-loaded O-GNR-PEG-DSPE showed about 78% viability compared to untreated controls, although this difference was not statistically significant.

### PrestoBlue® Assay to Assess Cell Death

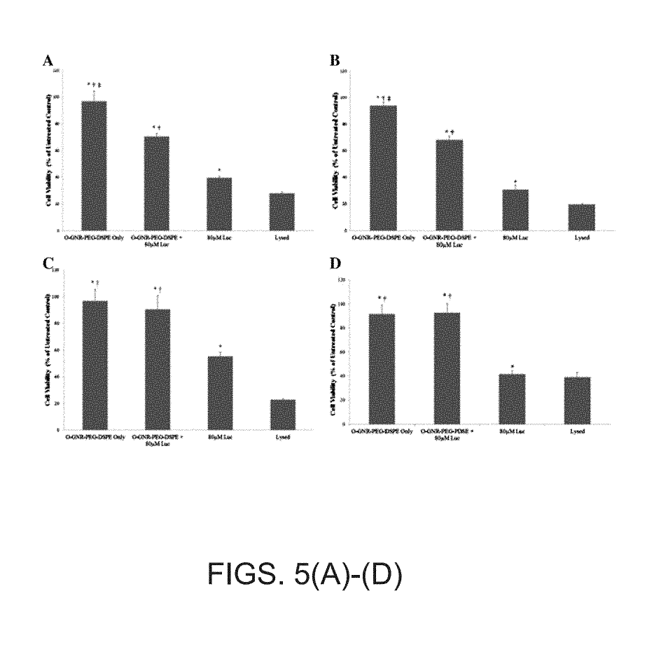

PrestoBlue® is a resazurin dye based cell viability assay in which healthy cells with normal metabolic activity and mitochondrial integrity can convert resazurin in the assay reagent to a pink fluorescent dye, with the amount of nonfluorescence to fluorescence conversion being directly proportional to the number of healthy cells present. FIGS. 5A and 5B illustrate that CMV/U251 and A1-5/CMV/U251 show a viability of about 40% and about 30% compared to untreated controls when exposed to 80 μM of free Luc which changes to about 70% viability for both cell lines when the drug is loaded onto O-GNR-PEG-DSPE. FIGS. 5C and 5D show that compared to untreated cells, CG-4 and MCF-7 do not show statistically significant toxicity when exposed to 80 μM Luc loaded O-GNR-PEG-DSPE.

### Annexin V/PI Staining of U251 Cells Exposed to Free or Loaded Drug

Annexin V (AV) is a 35 KDa phospholipid binding protein with an affinity for phosphotidyl serine. Early apoptotic cells show exposed phosphotidyl serine on their cell surface while it is still intact. PI on the other hand can only enter dead cells or early necrotic cells with compromised membranes. Thus, in flow cytometry AV+/PI+ represents dead cells, AV+/PI− represents cells in early apoptosis, AV−/PI+ represents dead or necrotic cells with compromised membranes, and AV−/PI− represents healthy living cells. Flow cytometry of CMV/U251 exposed to 5-80 μM Luc showed a concentration dependent decrease in AV−/PI− cells with no healthy living cells remaining after 4 hours of exposure to 80 μM of the drug. However, 37% of cells were still living after 4 hours of exposure to O-GNR-PEG-DSPE loaded with 80 μM Luc. APE-1 overexpressing A1-5/CMV/U251 also showed a concentration dependent decrease in cell viability with 100% cell death occurring at 40 μM exposure for 4 hours. However, exposure of these cells to Luc loaded nanoparticles showed 56% and 43% of the exposed cells were still living after 4 hours of exposure to O-GNR-PEG-DSPE loaded with 40 μM and 80 μM Luc respectively, the results are shown in Table 1 below.

TABLE 1
Toxicity of lucanthone is reduced when coated on graphene
nanoribbons (O-GNR) in GBM cell line U251.

| | Lucanthone | | | | % |
|---|---|---|---|---|---|
| Cell type | (μM) | % PI−AV− | % PI+ | % AV+ | PI + AV+ |
| CMV | C | 79 | 10 | 4 | 6 |
| | 5 | 20 | 78 | 0 | 17 |
| | 10 | 5 | 95 | 0 | 0 |
| | 20 | 3 | 97 | 0 | 0 |
| | 40 | 3 | 97 | 0 | 0 |
| | 80 | 0 | 95 | 5 | 0 |
| A1-5 | C | 72 | 15 | 3 | 10 |
| | 5 | 24 | 76 | 0 | 0 |
| | 10 | 8 | 92 | 0 | 0 |
| | 20 | 2 | 98 | 0 | 0 |
| | 40 | 0 | 100 | 0 | 0 |
| | 80 | 0 | 100 | 0 | 0 |
| CMV/GONR | C | 96 | 0 | 0 | 4 |
| | 5 | 84 | 12 | 3 | 1 |
| | 10 | 77 | 23 | 0 | 0 |
| | 20 | 74 | 26 | 0 | 0 |
| | 40 | 52 | 48 | 0 | 0 |
| | 80 | 37 | 63 | 0 | 0 |
| A1-5/GONR | C | 99 | 1 | 0 | 0 |
| | 5 | 77 | 23 | 0 | 0 |
| | 10 | 77 | 23 | 0 | 0 |
| | 20 | 76 | 24 | 0 | 0 |
| | 40 | 56 | 44 | 0 | 0 |
| | 80 | 43 | 57 | 0 | 0 |

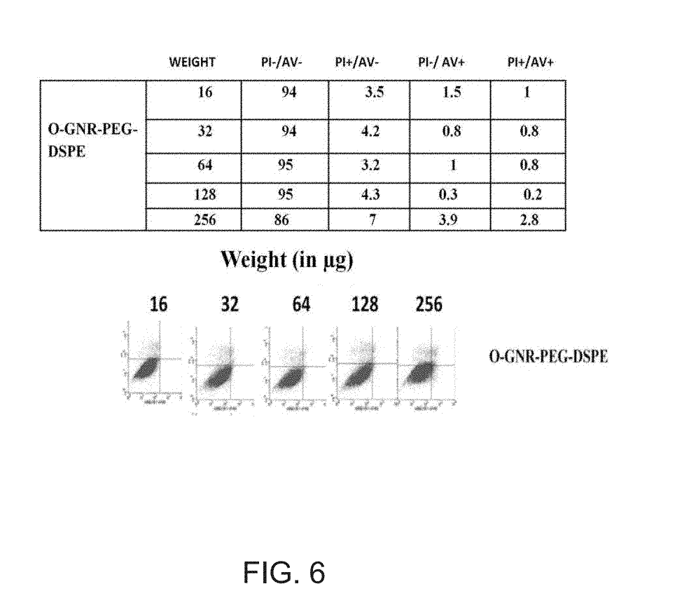

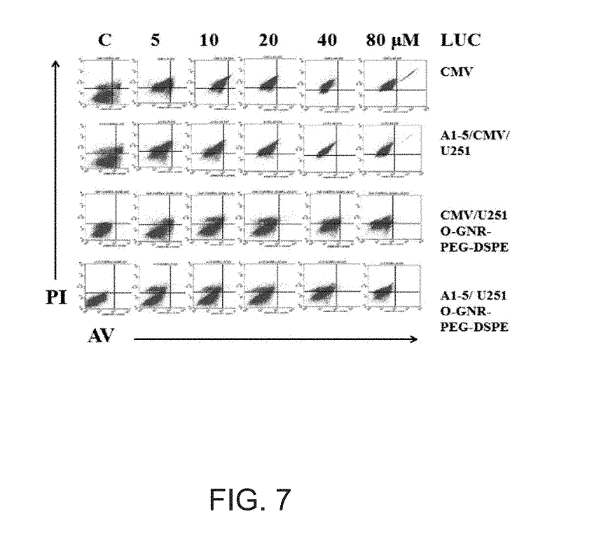

Exposure of the cells to different amounts of nanoparticles by themselves did not show significant toxicity either, as shown in FIG. 6. Flow cytometry data also indicated that dead cells were mainly PI+/AV− in nature, as illustrated in FIG. 7.

The efficient delivery of thioxanthanones like Luc and its analogues to GBM tumors for inhibition of overexpressed APE-1 includes a drug delivery agent that is relatively stable in aqueous solutions, loads relatively high concentrations of the drug, and can be taken up by the tumor cells. Also, there should be a relatively slow, sustained and controlled release of the drug so as to decrease the non-specific release of the drug before reaching its target site.

Up to about 310 μM of Luc could be loaded onto each milligram of O-GNR-PEG-DSPE. Other thioxanthanones (hycanthone and its structural analogue Inadazole-6 (IA-6)) with similar structure are expected to have similar loading efficiency on O-GNR-PEG-DSPE.

Flow cytometry based analysis indicates that about 67% of CMV/U251 and about 60% of A1-5/CMV/U251 cells showed presence of these particles after 24 hours proving that these particles get taken up by U251, as shown in FIGS. 3A-3I. In comparison, only about 38% of MCF-7 and about 29% of CG-4 showed presence of O-GNR-PEGDSPE in them indicating that these particles have a cell specific higher uptake in the GBM cell lines, as shown in FIGS. 3A-3I.

TEM images of CMV/U251 showed that these cells could take up large aggregates whereas MCF-7/CG-4 failed to do so resulting in accumulation of large O-GNR-PEG-DSPE aggregates on the surface, as shown in FIGS. 4A-4F. Uptake of large aggregates results in better drug delivery and hence more efficient human apurinic/apyrimidinic (AP) endonuclease (APE-1) inhibition in CMV/U251. PrestoBlue® assay of the four cell lines (U251, A1-5/U251, MCF-7 and CG-4) exposed to free Luc (80 μM) and Luc (80 μM) loaded on O-GNR-PEG-DSPE showed that loading decreased the toxicity of Luc by about 30% in U251 cells and about 40% in APE-1 overexpressing U251 cells compared to free drug, as shown in FIGS. 5A-5D. However, the cell death observed in the drug loaded nanoparticles was significantly higher compared to unexposed cells, as shown in FIGS. 5A-5D. Luc loaded O-GNR-PEG-DSPE did not show high toxicity in CG-4 and MCF-7 cells although the free drug was toxic to them, due to lower uptake of Luc-O-GNR-PEG-DSPE uptake in these cells, as shown in FIGS. 3A-3I.

This differential uptake also applied when MCF-7 and CMV/U251 cells were exposed to Luc-O-GNR-PEG-DSPE together as co-culture. Although CMV/U251 exposed to 80 μM Luc loaded on O-GNR-PEG-DSPE exhibited only about 56% viability compared to untreated control, MCF-7 exposed to Luc-loaded O-GNR-PEG-DSPE in the same wells as CMV/U251 did not show significantly reduced viability compared to untreated controls, as shown in FIG. 4E. This differential uptake and toxicity was further confirmed when CG-4 also showed much lower toxicity compared to CMV/U251 in coculture, as shown in FIG. 4F. Taken together, the TEM images and cell viability experiments indicate that U251 have higher uptake of drug loaded O-GNR-PEG-DSPE and hence shows higher cell death.

To test the mechanism of cell death induced by the drug loaded onto O-GNR-PEG-DSPE the affect of different concentrations (5-80 μM) of free Luc and Luc loaded on to nanoparticles was compared, when exposed for 4 hours to CMV/U251 and AI-5/CMV/U251 using Annexin V/PI staining. The U251 were sensitive to the free Luc with 100% cell death observed at 80 μM in CMV/U251 and 40 μM free drug concentration in AI-5/CMV/U251. The A1-5 overexpressor was more sensitive to free Luc likely due to about 35 fold higher expression of APE-1 in this overexpressor U251 clone.

Exposure of CMV/U251 to Luc loaded O-GNR-PEG-DSPE showed an increase in cell viability by about 64% at 5 μM and about 37% at 80 μM, as shown in FIGS. 5A-5D. Similarly, APE-1 overexpressing AI-5/CMV/U251 showed an increase in cell viability by about 53% at 5 μM and about 43% at 80 μM, as shown in FIGS. 5A-5D, of loaded drug compared to free drug at the same concentrations.

The toxicity of various weights of O-GNR-PEG-DSPE used to load 5-80 μM Luc on CMV/U251 was also compared. O-GNR-PEG-DSPE showed negligible toxicity at all weights tested, showing that the toxicity observed was primarily due to the released Luc. This also indicated that loading Luc onto O-GNR-PEG-DSPE does not affect its activity and thus the decrease in cell death may be due to the lower uptake of particles or slower release of Luc from O-GNR-PEG-DSPE. To ensure this differential toxicity observed is not due to lesser uptake of the O-GNR-PEG-DSPE particles, the toxicity based drug delivery experiments on Henrietta Lacks (HeLa) cells were repeated.

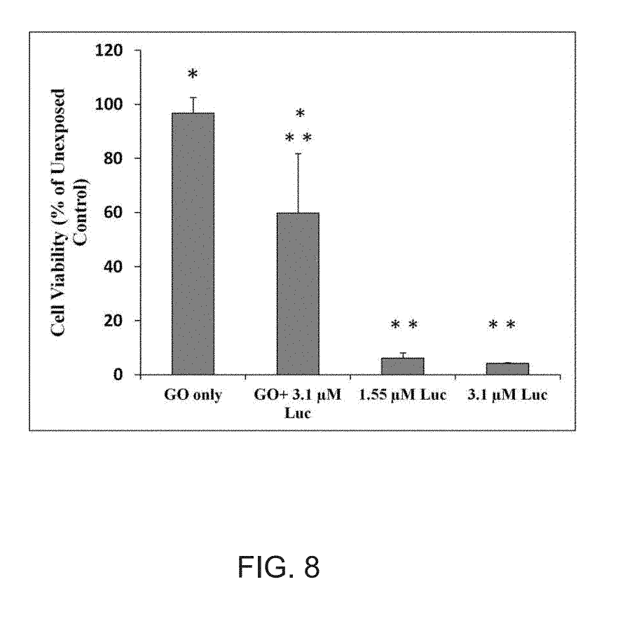

Analysis of PrestoBlue® cell viability assay showed that cell death observed after 24 hours of exposure to 3.1 μM of Luc loaded onto O-GNR-PEG-DSPE was significantly lesser than both 1.55 μM (50% of loaded) and 3.1 μM (same as loaded) of free drug showing that the decrease in toxicity observed was not due to low uptake of nanoparticles, as shown in FIG. 8. The decrease in cell death observed in drug loaded O-GNR-PEG-DSPE is likely due to slower release of the drug from the particles.

Another observation from Annexin V/PI results was that almost all the dead cells exposed to either free drug or drug loaded onto O-GNR-PEG-DSPE were PI+/AV− suggesting that the cell death was necrotic, as shown in FIG. 7.

### Example 2

Each of the delivery methods and systems discussed herein can be delivered to a subject in any suitable way, including intravenously and through targeted injections.

Efficient gene delivery and transfection using non-viral vectors remains a challenge due to the inability of these vectors to enter the nucleus. Unfunctionalized oxidized graphene nanoribbons (O-GNR's), at potential therapeutic doses are less cytotoxic than current widely employed non viral gene delivery vectors (Polyethyleneimine and Fugene 6®) and can deliver loaded genes to the nuclei of cells that have not undergone cell division. The O-GNR-plasmid DNA complexes are uptaken in large quantities into vesicular structures in HeLa and Human umbilical vein endothelial cells (HUVEC), and released inside the cell. The escaped O-GNR-DNA complexes potentially induce nuclear invaginations and penetrate the nuclear membrane to enter the nuclei. Gene delivery experiments showed that O-GNR's loaded with enhanced green fluorescence protein (EGFP) plasmid and siRNA against glyceraldehyde-3-phosphate dehydrogenase (GAPDH) show a concentration of treatment dependent increase in protein expression/inhibition, although, transfection efficiency after 12 hours was high (96-98%) for all concentrations tested in HeLa and HUVEC cells. These results are discussed below.

Graphene-based nanoparticles, such as Graphene oxide (GO) or graphene oxide nanoplatelets (GONPs), oxidized graphene nanoribbons (O-GNRs) and Graphene nano-onions (GNOs), can be used in various biomedical applications like drug delivery and imaging. O-GNR's derived from multiwalled carbon nanotubes (MWCNT's) have more oxidation groups on their surface and edges compared to GO leading to a lower (C/O) ratio.

As discussed below, O-GNRs without the need of additional functionalization serve as a versatile platform to load relatively large amounts of small or large sized genetic material (DNA or siRNA) and deliver them into the nucleus of cells to facilitate relatively high transfection efficiencies.

An oxidative method was employed to synthesize the oxidized graphene nanoribbons (O-GNR's) from MWCNTs (Sigma Aldrich, New York). 300 mg of MWCNTs were dissolved in concentrated sulphuric acid (60 mL) for 2 hours and 1.5 μm of potassium permanganate (KMnO₄) was added to the dispersion. This mixture was stirred for 60 minutes at room temperature following which it was heated, on an oil bath, to 55° C. for 45 minutes and an additional 15 minutes at 70° C. The mixture was cooled to 25° C., and washed with water and dilute aqueous hydrochloric acid. The O-GNR nanoparticles were floculated via addition of ethanol and ether, and separated from the dispersion by centrifugation at 3000 rpm for 30 minutes. The O-GNR pellet was dried overnight in an oven (at 60° C.) before being used.

Henrietta Lacks (HeLa) cells, MRC5 fibroblasts derived from normal lung tissue and Human umbilical vein endothelial cells (HUVEC) were obtained from ATCC (Manassas, Va., USA). HeLa and MRC5 cells were grown in Dulbecco's modified eagle's medium (DMEM) with 10% fetal bovine serum. 1% penicillin-streptomycin was used as antibiotic. HUVEC cells were grown in F-12K medium supplemented with 10% FBS, 100 μg/ml heparin and 30 μg/ml endothelial cell growth supplement. All cell lines were incubated at 37° C. in a humidified atmosphere of 5% CO₂, and 95% air.

### Cell Synchronization.

Unsynchronized HeLa and HUVEC cells were blocked at the G2/M stage of growth by treatment with nocodazole followed by release to obtain cellular synchronization. Briefly, 2×10⁵ cells were plated in 75 cm² flasks and allowed to grow for 24 hours in normal media. The media was then removed and the cells were washed with phosphate buffered saline. Growth medium containing 200 ng/mL nocodazole was added to the flask and the cells were allowed to grow in it for 30 hours. After 30 hours the media was removed, cells were collected by trypsinization and centrifuged at 500 g three times accompanied by resuspension of the cell pellet with fresh media after every centrifugation. The cell suspensions were then plated in 75 cm² plates and allowed to move into G1 phase together. This process was repeated several times to obtain ˜90% synchronized cells for each cell line. Post synchronization, HUVEC cells had a doubling time of ˜27 hours and HeLa cells ˜20 hours. All experiments were done with synchronized cells.

### Transmission Electron Microscopy

Transmission electron microscopy (TEM) was performed on O-GNR samples prepared by dispersing 1 mg of the particles in a water-ethanol mixture (1:1) mixture by probe sonication for 2 minutes (Cole Parmer Ultrasonicator LPX, 750 W, 2 sec on and 1 sec off cycle). The O-GNR suspension was centrifuged at 3000 rpm for 3 minutes. The supernatant after centrifugation was dropped onto holey lacey carbon grids on a copper support (Ted Pella, Inc., Redding, Calif.). TEM of these grids were performed using a Tecnai Bio Twin G transmission electron microscope (FEI, Hillsboro, Oreg.), at 80 kV. Digital images were acquired using an XR-60 CCD digital camera system. (AMT, Woburn, Mass.).

### Gene Loading

A 1.2 mg/ml dispersion of O-GNR was obtained by bath sonicating (Ultrasonicator FS30H, Fischer Scientific, Pittsburgh, Pa.) 4.8 mg of O-GNR in 4 mL phosphate buffered saline (PBS) for 30 minutes. Post sonication, 1 μg of enhanced green fluorescent protein (EGFP) plasmid (Addgene) or 50 μM siRNA against Glyceraldehyde 3-phosphate dehydrogenase (GAPDH) was added to the dispersion and stirred for 60 minutes on an ice bath. After the stirring, O-GNR loaded with EGFP or siRNA was centrifuged at 3000 rpm for 30 minutes in a cold centrifuge (4° C.) and the plasmid or siRNA left in the supernatant was quantified using a Nanodrop ND-100 (Nanodrop Technologies, Wilmington, Del.). This “loading” occurs due to a non-covalent interaction such as a van der Walls interaction. The plasmid loaded onto the O-GNR's was calculated by subtracting DNA or siRNA left in the supernatant from the total genetic material added. The EGFP or siRNA loaded O-GNR pellet was resuspended in DMEM at 100 μg/mL, 200 μg/mL, 400 μg/mL and 600 μg/mL.

O-GNR Tagging with FITC

5 mg of solid O-GNRs were suspended in 5 mL of FITC-PEG-DSPE (fluorescein isothiocyanate-1,2-distearoyl-sn-glycero-3-phosphoethanolamine-N-[amino(polyethylene glycol)]) solution in PBS and bath sonicated (Ultrasonicator FS30H, Fischer Scientific, 250 W, Pittsburgh, Pa.) for 15 minutes. Following sonication, the suspension was stirred at room temperature for 4 hours. Next, the suspension was centrifuged at 3000 rpm for 30 minutes. The supernatant was discarded and the pellet containing O-GNR-PEG-DSPE-FITC particles were resuspended in DMEM to achieve a concentration of 400 μg/mL.

### Toxicity Analysis Using LDH Assay

5×10³ MRC5 cells were plated per well in a 96 well plate and grown for 18 hours. Following this incubation, media was removed from the wells, and replaced with 180 μL of fresh media. Cytotoxicity of O-GNR (at 100 with 180 μg/mL), Fugene® 6 and PEI were then compared using a lactate dehydrogenase assay. To the fresh media, 20 μL of O-GNR at 1 mg/mL was added to give a final concentration of 100 μg/ml in solution. Fugene® 6 was added to the cells according to manufacturer's instructions (24 μL of the agent was added to 200 μL of media and 20 μL of this solution was added to each well). 20 μL of 5 μg/mL PEI (branched, 25 kD, Sigma-Aldrich), was added to each well to give a final concentration of 1 μg/mL in solution. The cells along with transfection agents were incubated for 72 hours. After 72 hours the 96 well plates were centrifuged at 1200 rpm for 5 minutes. 50 L of media from each well of the centrifuged 96 well plate was removed and added to a fresh 96 well plate. 100 μL of LDH reagent (Tox7 reagent, Sigma-Aldrich) was added to each well and incubated for 45 minutes.

Absorbance readings of the plate were taken in a BIOTEK ELx 800 absorbance micro plate reader at 490 nm. Untreated cells were treated as control. Lysed control was prepared by adding 20 μL of lysis solution to the untreated cells, 45 min before centrifugation of the plate. The LDH release (% of lysed control) is expressed as the percentage of (ODₜₑₛₜ−OD_{blank})/(OD_{lysed}−OD_{blank}), where ODₜₑₛₜ is the optical density of the control cells, or cells exposed to gene delivery agents, OD_{lysis} is the optical density of the positive control cells, and OD_{blank} is the optical density of the wells without cells.

### Confocal Microscopy

25×10³ HeLa and HUVEC cells were plated per well in glass bottom confocal plates and grown for 18 hours. Following this incubation, media was removed and replaced with 360 μL of fresh media. 40 μL of either O-GNR loaded with EGFP at the three different concentrations (200 μg/mL, 400 μg/mL and 600 μg/mL) or O-GNR-PEG-DSPE-FITC at 400 μg/mL was added to each plate. The plates were incubated for 30 minutes or 12 hours following which EGFP loaded O-GNR particles or O-GNR-PEG-DSPE-FITC were washed away using PBS washes and the cells were fixed in 2.5% glutaraldehyde (Electron Microscopy Sciences, Hatford, Pa.) for 30 minutes. Hoescht 3342 stain (Invitrogen) was used to stain the nuclei of cells. Images were obtained using a Zeiss LSM 510 META NLO Two-Photon Laser Scanning Confocal Microscope System.

### Assay for GAPDH Activity

GAPDH activity was assayed using KDalert™ GAPDH Assay Kit (Life Technologies, Foster City, Calif.). 5×10³ HeLa cells per well were plated in 96 well plates and grown for 18 hours. This was followed by removal of media from the wells and addition of 180 μL of fresh media. 20 μL of O-GNR's loaded with siRNA at various concentrations (100 μg/mL, 200 μg/mL, 400 μg/mL and 600 g/mL) was then added to the wells to achieve final treatment concentrations of 10 g/mL, 20 μg/mL, 40 μg/mL and 60 μg/mL respectively and incubated for 48 hours.

Following this incubation, the media containing the loaded O-GNR's was removed and the cells were then treated with 200 j L cell lysis buffer provided with the kit for 20 minutes. Post cell lysis 10 μL of the lysate from each well was moved to a fresh 96 well plate. 90 μL of the diluted master mix from the kit was added to the wells and the fluorescence in the wells was measured using an Infinite M200 multiwell plate reader (Tecan Group, Morrisville, N.C.) at 545 nm excitation and 575 nm emission after 5 minutes. Untreated cells and cells treated with O-GNR's (60 μg/mL) loaded with negative control siRNA were used as controls.

### Transmission Electron Microscopy of Cells

25×10⁴ HeLa and HUVEC cells were plated per well in 6 well plates covered with ACLAR® film (Electron Microscopy Sciences, Hatford, Pa.) and grown for 18 hours. Following this incubation the media was removed and replaced with media containing O-GNR at 20 μg/mL. The cells were incubated in the media for 30 minutes or 12 hours after which the excess O-GNR was washed away using PBS. The films containing cells were either trypsinized for cell counting or fixed with 2.5% glutaraldehyde (Electron Microscopy Sciences, Hatford, Pa.). The fixed films were then placed in a solution of 2% Osmium Tetroxide (in PBS) given ethanol washes and embedded in durcupan resin (Sigma-Aldrich, St. Louis, USA). Cells on the film were then blocked, cut into thin sections (about 80 nm) using an Ultracut E microtome (Reichert-Jung, Cambridge, UK), and placed on copper grids coated with formavar. This obtained cell sections were viewed with a Tecnai Bio Twin G transmission electron microscope (FEI, Hillsboro, Oreg.), at 80 kV. Digital images were acquired using an XR-60 CCD digital camera system. (AMT, Woburn, Mass.)

### Flow Cytometry

5×10⁴ HeLa cells were grown in 10 cm dishes for 20 hours. Following this incubation, one dish was trypsinized and the cell number was determined. Media was removed and replaced with 9 mL of fresh media in the other dishes. O-GNR's were loaded with propidium iodide (PI) by sonicating the particles (5 mL at a concentration of 1 mg/mL) with 10 mg PI and stirring the mixture for 4 hours at room temperature. The stirred mixture was centrifuged at 3000 rpm for 30 minutes. The supernatant was discarded and the pellet was resuspended in DMEM to give O-GNR-PI at a concentration of 400 μg/mL. The HeLa cells were treated with 1 mL of O-GNR-PI to give a final concentration of 40 μg/mL in solution. Untreated cells were used as control. After 2, 12 and 24 hrs, cells were trypsinized, resuspended in FACS buffer (Phosphate buffered saline containing 20% fetal bovine serum) and placed on ice. Flow cytometry to detect PI fluorescence was performed on all samples using a FACS Calibur Cell Sorter (BD Biosciences, San Jose, Calif.). Cell number was determined at all time points tested to determine if cell division had taken place.

### Results: Transmission Electron Microscopy

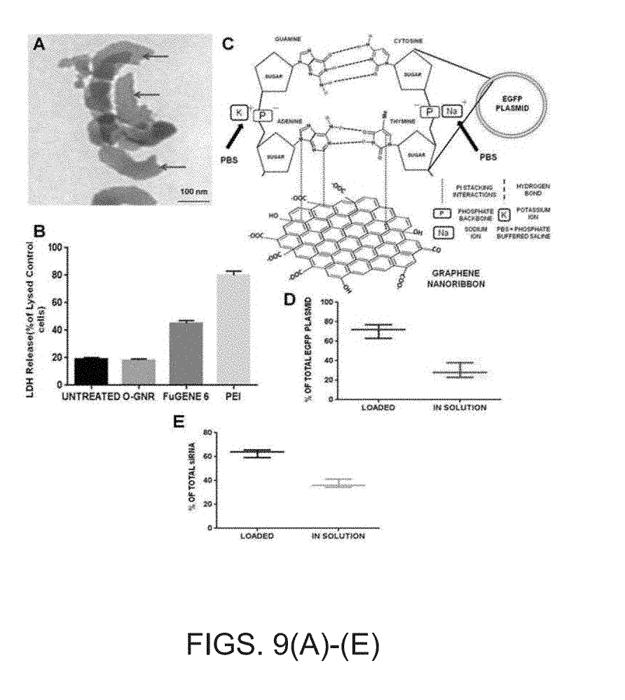

FIG. 9A shows representative low resolution TEM image of multiple O-GNRs. The figure shows O-GNR's approximately 300-500 nm in length and 80-125 nm in breadth (arrows) having a relatively high aspect ratio (Ratio of length to breadth is >5). The images indicate that O-GNR's have a large flat surface area that can allow loading of a volume of genetic material.

### Results: Cytotoxicity on Normal Cells

Using the LDH assay, cytotoxicity of 100 g/mL O-GNR's after 72 hours of incubation with normal lung fibroblasts (MRC5) was compared to that of the maximum suggested dose of two other commonly used transfection agents. FIG. 9B shows the cytotoxicity of O-GNR's and transfection agents Fugene 6® and PEI on MRC5 cells. Compared to untreated control, O-GNR's at 100 μg/mL did not show a significant increase in cell death. Incubation with Fugene 6® shows about 25% increase in LDH release from MRC5 cells compared to untreated control. Treatment with PEI shows about 60% increase in LDH release compared to untreated control.

Results: Gene Loading onto O-GNR's

FIGS. 9D and 9E show the amount of EGFP plasmid and siRNA that could be loaded onto the nanoparticles when incubated with 1.2 mg/mL O-GNRs for 60 minutes. FIG. 9D shows that about 74.5% of the starting weight of the EGFP (1000 ng) could be loaded onto the O-GNR (at 1.2 mg/ml). About 25.5% of the DNA stayed in solution after centrifugation. FIG. 9E shows that about 63% of the initial siRNA concentration (50 μM) could be loaded onto the nanoribbons whereas about 37% stayed in the supernatant after centrifugation. O-GNR's (tested of MRC5 fibroblasts) showed no evidence of toxicity up to about 100 μg/mL (FIG. 9C).

### Results: Transfection of EGFP

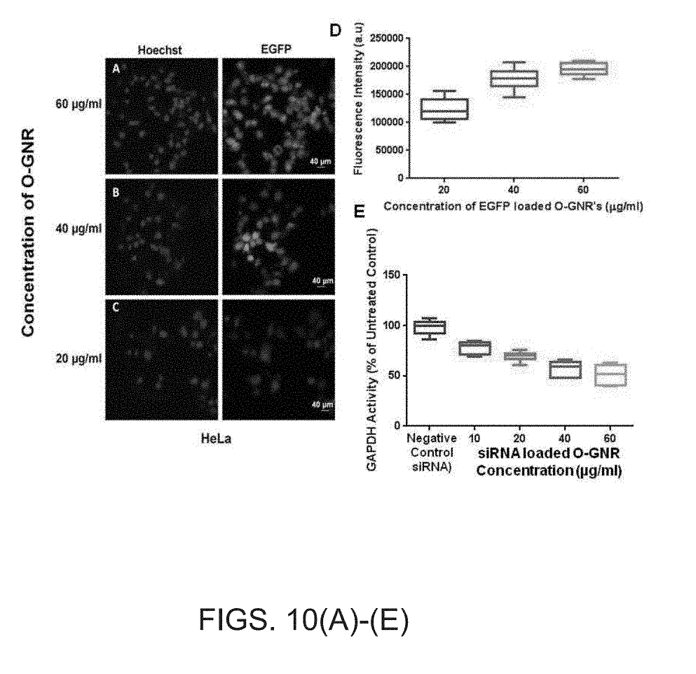

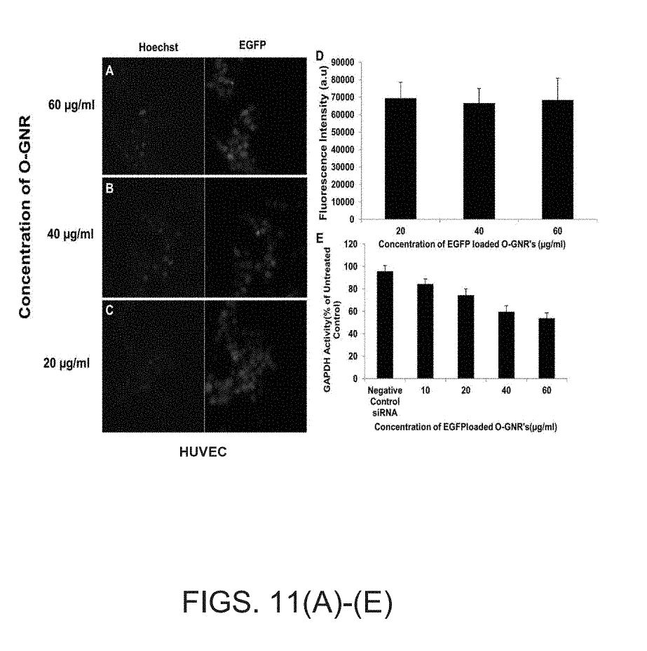

FIGS. 10A-10E and FIG. 11A-11E show the results from treatment of EGFP plasmid and siRNA (against GAPDH) loaded O-GNR's for 12 hours in HeLa and HUVEC cells respectively. FIGS. 10A-10C and FIG. 11A-11C shows nuclear staining and EGFP fluorescence of the treated HeLa cells exposed to EGFP loaded O-GNR at 20, 40 and 60 μg/mL. All cells in the representative images show green fluorescence due to expression of EGFP. FIG. 10D represents the fluorescence quantification of treated HeLa cells and shows a concentration dependent increase in fluorescence intensity with 60 μg/ml treated cells showing maximum fluorescence. However, HUVEC cells did not show the same response as observed in HeLa cells (FIG. 11D).

FIG. 10E shows the decrease in GAPDH enzyme activity in HeLa cells exposed to siRNA (against GAPDH) loaded O-GNR's at increasing concentrations (0-60 μg/mL) for 12 hours. The figure shows GAPDH activity decreases to about 78%, about 69%, about 57% and about 51% of untreated cells at treatment concentrations of 10, 20, 40 and 60 μg/mL loaded O-GNR's. However, cells treated with O-GNR's (at 60 μg/mL) without loaded siRNA did not show similar decrease. HUVEC cells showed similar response to siRNA loaded O-GNR's as observed in HeLa cells (FIG. 11E).

O-GNR's loaded with EGFP and siRNA showed a concentration dependent increase in fluorescence in HeLa cells and decrease in GAPDH activity in HeLa cells indicating that exposure to more O-GNR's leads to more EGFP plasmid and siRNA reaching the nucleus of the cells. However, gene delivery efficiency for all the concentrations (obtained by observing fluorescence in 300 cells exposed to each concentration) was relatively high (about 96-about 98%) (FIG. 10A-10E). HUVEC cells showed similar transfection efficiency although concentration of O-GNR dependent increase in fluorescence was not observed in them (FIG. 11A-11E).

Results: O-GNR Uptake into Cells

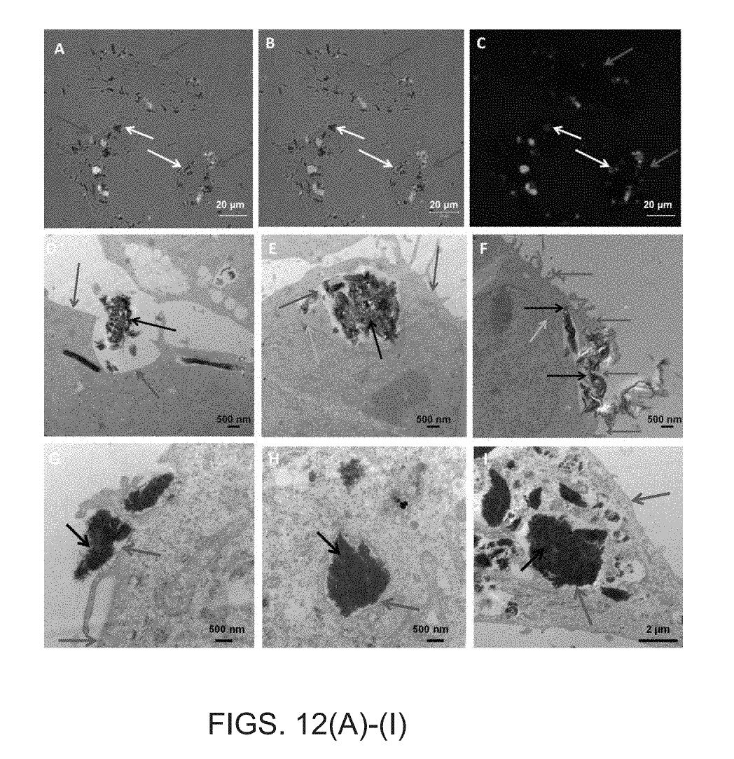

Entry of O-GNR into cells was confirmed by confocal microscopy and TEM. FIGS. 12A-12C are representative images of HeLa cells exposed to 40 μg/mL O-GNR-PEG-DSPE-FITC for 30 minutes. FIG. 12A shows the brightfield images of the cells (dark arrows) and O-GNR (light arrows). FIG. 12B shows the merge of both brightfield and fluorescence image showing co-localization of the O-GNR particles (in FIG. 12A) with the FITC fluorescence indicating the O-GNR (light arrows) was loaded with FITC-PEG-DSPE (fluorescence). FIG. 12C shows the fluorescence only image of O-GNR-PEG-DSPE-FITC (fluorescence). Overall, the images show that within 3 hours of incubation the O-GNR-PEG-DSPE-FITC particles are either inside the cells or in the process of being uptaken.

FIGS. 12D-12I are representative TEM images of HeLa and HUVEC cells treated with 20 g/mL O-GNR-PEG-DSPE-FITC for 30 minutes. FIGS. 12D-12F represent HeLa cells treated with O-GNRs whereas FIGS. 12G-12I are representative TEM images showing HUVEC cells treated with O-GNRs. FIG. 12D and FIG. 12G shows invagination of the cellular membrane (lighter arrows) near O-GNR (darker arrow) aggregates. FIGS. 12E and 12H show the formation of large vesicular structures (lighter arrow) containing O-GNR aggregates (darker arrow) inside HeLa and HUVEC cells exposed to the particles.

FIG. 12F shows the disruption of large portions of a HeLa cell membrane (lighter arrows) near the cell membrane in contact with O-GNR aggregates (darker arrows). FIG. 12I shows multiple O-GNR aggregate (darker arrow) filled vesicular structures (lighter arrows) in the cytoplasm of HUVEC cells. On an average, from 10 different TEM images for each exposed cell line, HUVEC cells showed higher amount of O-GNR uptake (about 23 aggregates/cell on average) compared to HeLa cells (about 7 aggregates/cell on average). Overall, the images show uptake of O-GNR aggregates into cells within 30 minutes of incubation. This uptake can involve invagination of the cell membrane as well as formation of protrusions on the membrane to take in the particles on the surface.

FITC-PEG-DSPE loaded O-GNR showed presence of O-GNR inside the cells after 30 minutes of exposure indicating a relatively quick uptake of the particles (FIG. 12A-12I). TEM images showed formation of large vesicles from the membrane of HeLa and HUVEC cells containing O-GNR aggregates indicating that unlike GO, DNA loaded O-GNR's are taken up into the cells (FIG. 12A-12I).

Results: O-GNR Escape from Intracellular Vesicles

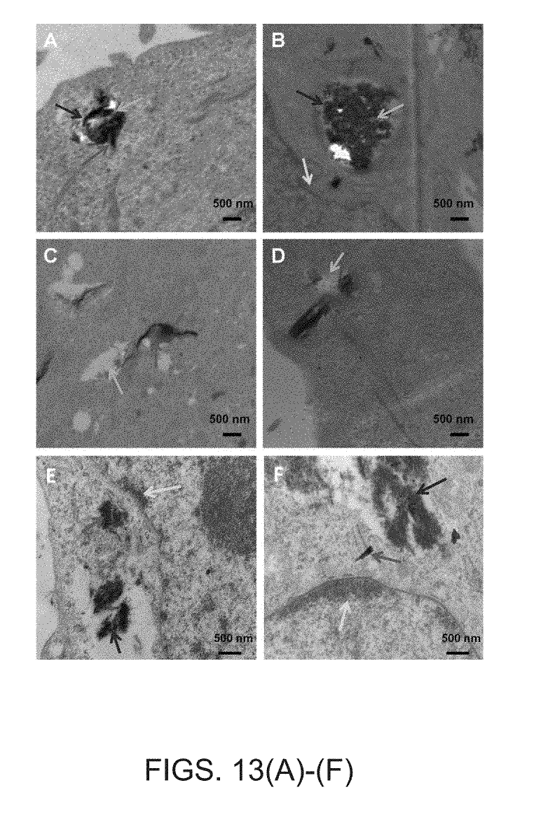

TEM images were used to analyze if O-GNR could potentially escape from vesicular compartments they were uptaken into as they enter the cells. FIGS. 13A-13D are representative TEM images of HeLa cells incubated with 20 μg/mL O-GNR's for 12 hours. FIG. 13A shows an O-GNR aggregate (darker arrows) outside a vesicle (lighter arrows) which has potentially escaped through membrane penetration. FIG. 13B shows an O-GNR aggregate (darker arrow) which has potentially escaped a large vesicle containing O-GNR aggregates (lighter arrow) at the nuclear periphery. Invagination of the nucleus is also observed in the figure (lightest arrow). FIG. 13C shows two O-GNR aggregates (darker arrows) near lysed vesicles (lighter arrows). FIG. 13D also shows a large O-GNR aggregate (darker arrow) near a lysed vesicle (lighter arrow). FIG. 13E-13F are representative TEM images of HUVEC cells incubated with 20 μg/mL O-GNR's for 12 hours). Both figures show presence of individual O-GNR aggregates (darker arrows) outside aggregate containing vesicular structures. A dark region in the nucleus (lighter arrows) near the released nanoparticles is also observed. This dark region was not observed in the HeLa cells.

TEM images of HeLa cells treated with O-GNR's for 12 hours indicated that O-GNR's can escape endosomes or can lyse them to escape into cytoplasm (FIGS. 13A-13F).

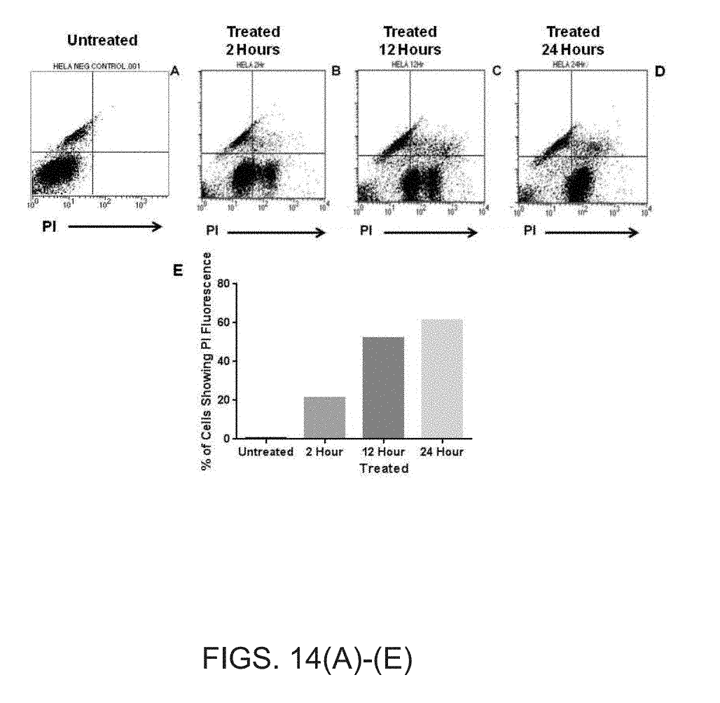

Flow cytometry was used to estimate the efficiency and time taken for vesicular escape of O-GNR in HeLa cells treated with PI loaded O-GNR for 2-24 hours. FIGS. 14A-14D show the number of HeLa cells that showed presence of PI fluorescence in its nucleus out of 10⁴ cells analyzed. FIGS. 14A-14D show the distribution of fluorescent and non-fluorescent cells after 2-24 hours of treatment using flow cytometry and FIG. 14E shows the corresponding quantification of the data. FIGS. 14A and 14D shows that about 21% cells incubated with O-GNR-PI for 2 hours showed PI fluorescence. FIGS. 14B and 14D shows that about 51% cells incubated with O-GNR-PI for 12 hours showed PI fluorescence. FIGS. 14C and 14D shows that about 60% cells incubated with O-GNR-PI for 24 hours showed PI fluorescence in the nucleus.

Live cells exposed to PI loaded O-GNR at a previously calculated non-toxic concentration of O-GNR would only show fluorescence before the cells divide if the O-GNR can get into the cells, escape the vesicles and enter the nucleus. Flow cytometry based analysis of HeLa cells exposed to PI loaded O-GNR showed that about 21% and about 51% cells showed PI fluorescence in the nucleus after 2 and 12 hours (before the cells have divided, cell counts shown in FIGS. 11A-11E) and about 60% of the cells showed PI fluorescence after 24 hours (cell counts shown in FIGS. 11A-11E). This indicates that about 50% of cells showed endosomal escape of O-GNR, release of PI from it and nuclear translocation of either already released PI or PI loaded O-GNR within 12 hours (FIGS. 14A-14E).

Results: O-GNR Entry into Nucleus

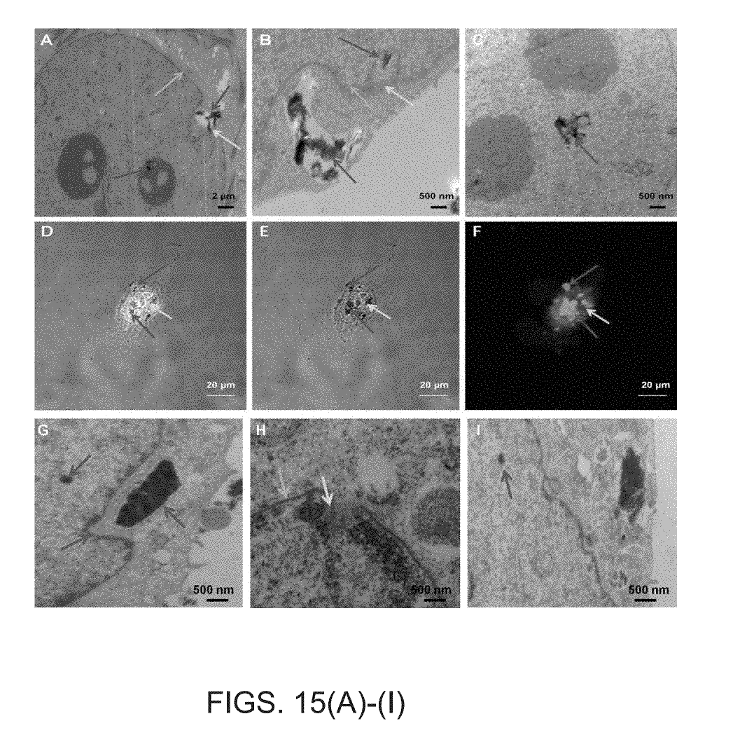

Entry of O-GNR into nucleus was confirmed by TEM and confocal microscopy studies with HeLa and HUVEC cells. FIGS. 15A-15C shows representative TEM images of HeLa cells treated with 20 μg/mL O-GNR for 12 hours. FIG. 15A is a representative TEM image of a HeLa cell showing a O-GNR aggregate (darker arrow outside nucleus, nuclear periphery demarcated by lighter arrow) likely escaping from an O-GNR containing vesicle and formation nuclear invaginations (lightest arrows) near the vesicle. The image also shows O-GNR aggregates both inside and outside the nucleus (dark arrows). FIG. 15B shows representative TEM image of a HeLa cell uptaking O-GNR inside the cell (darker arrow outside nucleus, nuclear periphery demarcated by lighter arrow). The image also shows O-GNR aggregates inside the nucleus (dark arrows) and formation of nuclear invaginations. From the image it can also be observed that the nuclear membrane is closing around the nuclear invaginations to potentially take up the O-GNR aggregate. FIG. 15C shows a representative TEM image of HeLa cell nuclei (lighter arrow) which shows presence of O-GNR aggregates (dark arrow). No vesicular structures were observed associated with the particles inside the nuclei.

FIGS. 15D-15F shows bright field, nuclei fluorescence and merge of nuclei and FITC fluorescence images of a HeLa cell treated with 40 μg/mL FITC-PEG-DSPE-O-GNR for 12 hours. FIG. 15D shows presence of O-GNR aggregates (dark arrows) in and around the nucleus (light arrow). FIG. 15E shows the same cell with nuclei stain (Hoechst stain, light arrow). FIG. 15F shows the same cell with green fluorescence from the FITC-PEG-DSPE loaded O-GNR. The fluorescence co-localizes with the O-GNR aggregates (dark arrows) showing proper loading of the FITC-PEG-DSPE on O-GNR. The particles are mostly localized inside the nucleus (demarcated by blue fluorescence)

FIGS. 15G-15I shows representative TEM images of HUVEC cells treated with 20 μg/mL O-GNR for 12 hours. FIG. 15G shows a large O-GNR aggregate containing vesicle (darker arrow) and nuclear invagination similar to that observed in HeLa cells (lighter arrow). Darkening of the nucleus near the O-GNR aggregate is observed as also seen in FIGS. 13A-13F. An O-GNR aggregate is also observed inside the nucleus (darkest arrow). FIG. 15H shows the opening of the nucleus (gap in the nucleus indicated by lightest arrows, nucleus indicated with lighter arrows) in presence of O-GNR aggregates potentially released from vesicles (red arrows). The opening of the nucleus is seen to occur at a dark spot (darker arrow) on the nucleus near the O-GNR aggregates. FIG. 15I shows the absence of dark spots in the nuclear membrane (lighter arrow) without any O-GNR aggregates in the vicinity. An O-GNR aggregate is seen inside the nucleus (darker arrows) in the image.

Dark regions in the nuclear membrane observed in several TEM images of HUVEC cells (FIGS. 12E-12F, FIGS. 15G-15H) are generally associated with heterochromatin. Heterochromatin has been known to be associated with actin related proteins that influence actin organization in the nucleus. Formation of heterochromatin rich structures near the O-GNR's can indicate that the O-GNR's can influence the distribution of actin on the nuclear membrane to induce formation of the invaginations. In some images, it was also observed that post formation of nuclear invaginations and entry of O-GNR's into them, the nuclei membrane reforms around the invaginations (FIG. 15B).

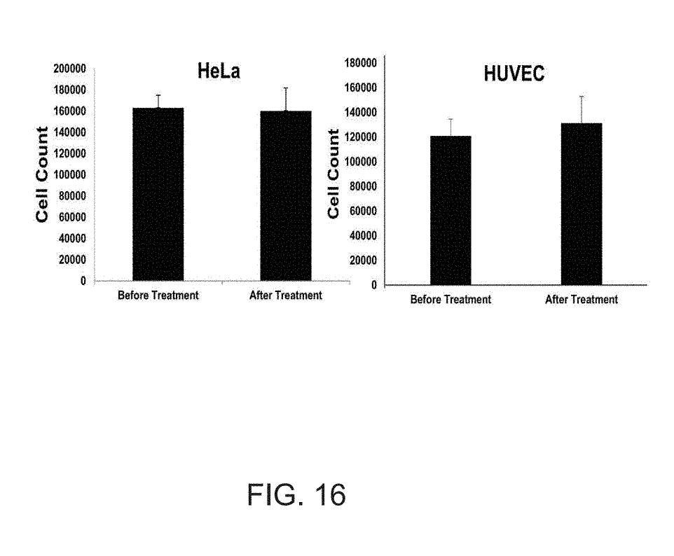

To elucidate if O-GNR could enter the nucleus TEM images were used of cells exposed to O-GNR for 12 hours. Nuclear entry of O-GNR was viewed by confocal microscopy of HeLa and HUVEC cells exposed to FITC-PEG-DSPE loaded O-GNR for 12 hours (cell counts before and after 12 hour incubation shown in FIG. 16). Results from both TEM and confocal studies showed presence of O-GNR inside the nucleus.

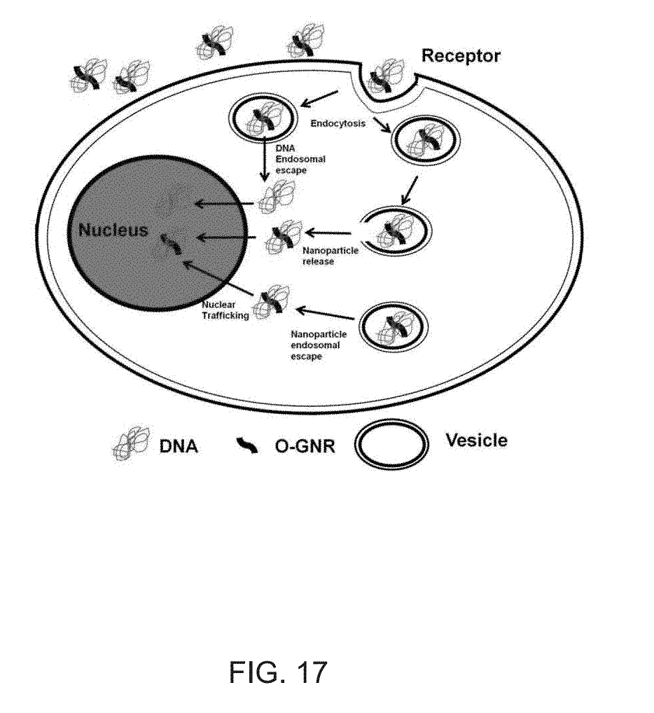

Post uptake, the O-GNR's can undergo vesicular escape either by penetrating the vesicular membrane or by lysing the vesicles (FIG. 17). Escaped O-GNR's induce formation of nuclear invaginations that can lead to entry of the particles into the nucleus (FIG. 17).

This example demonstrates that O-GNR's prepared by oxidative unzipping of MWCNT's are non-toxic to fibroblasts at a concentration of 100 μg/mL and can load relatively high amounts of dsDNA and siRNA onto them in a solution of phosphate buffered saline. The loaded DNA/siRNA can be delivered to cells with a concentration of treatment dependent expression of the delivered gene, e.g. higher incubation concentrations resulted in more dsDNA reaching the nucleus and hence more expression of the protein and a concentration dependent decrease in GAPDH activity was also observed. For the concentrations tested (20-60 μg/mL) transfection efficiency for O-GNR was between about 96-about 98% in HeLa and HUVEC cells.

### Example 3

### Materials

In this example, C₂₄ and C₁₆ ceramide and C₁₂ NBD ceramide were purchased from Avanti Polar Lipids (Alabaster, Ala., USA). DSPE-PEG was purchased from NOF America Corporation (White Plains, N.Y., USA). All other materials and reagents were purchased from Sigma Aldrich (St. Louis, Mo., USA) unless otherwise noted.

### O-GNR and GNP Synthesis

O-GNRs were synthesized from multi-walled carbon nanotubes (MWCNTs) using the longitudinal unzipping method with centrifugation instead of filtration for purification. MWCNTs (150 mg) were dispersed in 30 mL of concentrated sulfuric acid. After 3 hr, 750 mg of potassium permanganate was added and then mixture was stirred for 1 hr. The mixture was then heated to approximately 60° C. for 1 hr in an oil bath to complete the reaction. It was then allowed to cool to room temperature, and was washed with dilute hydrochloric acid. The product was isolated by flocculation using ethanol and ether, followed by centrifugation at 3000 rpm and drying overnight in a vacuum oven.

Loading of Ceramide onto Carbon Nanoparticles

Stock solutions of O-GNRs, C₂₄ and C₁₆ ceramide, and PEG-DSPE were dispersed in 100% ethanol at a concentration of 1 mg/mL using a bath sonicator. 200 μL of each solution were added to a 20 mL glass scintillation vial, and the volume was made to 1 mL using 100% ethanol. This mixture was bubbled with nitrogen gas and covered in parafilm to prevent oxidation of ceramide. It was then bath sonicated for 15 minutes to allow interspersion of individual nanoparticles and ceramide molecules.

To this solution, 9 mL of double-distilled water were added using a New Era NE-300 syringe pump (New Era Pump Systems Inc. Farmingdale, N.Y., USA) over a period of one hour under constant sonication. In order to separate ceramide-loaded nanoparticles from unloaded ceramide, the mixture was centrifuged at 13000 rpm for at least 30 minutes. The supernatant was discarded and the nanoparticles were washed by resuspension in pure double-distilled water and repeating the centrifugation step, yielding ceramide-loaded nanoparticles. For sham controls, C₁₆ or C₂₄ ceramide underwent the loading process without any nanoparticles (volume replaced by 100% ethanol). For O-GNR controls, O-GNRs underwent the loading process without ceramide (volume replaced by 100% ethanol).

### Cell Culture

HeLa cells were obtained from ATCC (Manassas, Va., USA). Cells were grown in Dulbecco's Modified Eagle Medium (DMEM) supplemented with 10% fetal bovine serum and 1% penicillin-streptomycin. Cells were grown at 37° C. in a humidified atmosphere of 5% carbon dioxide.

### Viability and Apoptosis Assays

In order to assess the ability of C₂ ceramide to prevent damage to cells, and C₁₆ ceramide to sensitize cells to other treatments, HeLa cells were seeded in flat-bottomed 96 well plates at a density of 5000 cells/well and were allowed to adhere overnight. For cell assays, plates were either subjected to treatment with free C₆ ceramide or UV irradiation. Immediately following C₆ or UV irradiation, C₁₆ and C₂₄ Ceramide-loaded O-GNRs were added to each well at various concentrations ranging from 5-40 pig/mL of nanoparticles. After 24 hours, the media was aspirated and each well was rinsed twice with PBS. A mixture of 10 μL of Presto Blue Viability Reagent (Life Technologies, Grand Island, N.Y., USA) and 90 μL of DMEM with 10% FBS and 1% P/S were added to each well and placed back in the incubator. After 2 hours, the plates were removed and the fluorescence intensity of each well was measured using a Molecular Devices SpectraMax M2e (Sunnyvale, Calif., USA) with an excitation wavelength of 560 nm and an emission wavelength of 590 nm. Lysed cells were used as a positive control, and untreated cells were used as a negative control. The cell viability is expressed as a percent of the lysis control with the formula:

((Iₜₑₛₜ−I_{blank})/(I_{control}−I_{blank})×100%)−100%

where Iₜₑₛₜ is the fluorescence intensity of cells exposed to ceramide-loaded nanoparticles, I_{control} is the fluorescence intensity of lysed cells, and I_{blank} is the fluorescence intensity from empty wells.

Apoptosis was assessed at the highest levels of nanoparticles tested using a Biovision Caspase 3 Fluorometric Assay (Biovision, San Francisco, Calif.).

### Confocal Microscopy

C₁₂ NBD ceramide was loaded onto O-GNRs using the same procedure as C₂₄ and C₁₆ ceramide. HeLa cells were seeded in glass-inset confocal dishes at a density of 7.5×10⁴ cells/mL with 2 mL per plate and allowed to adhere overnight. Cells were treated with either 5 μM fluorescent C₁₂ NBD ceramide or an equivalent amount of fluorescent C₁₂ NBD ceramide loaded on O-GNRs for 2 hours and imaged using a Leica TCS SP8 Laser Scanning Confocal Microscope System (Buffalo Grove, Ill., USA). Cells were pretreated with inhibitors of O-GNR uptake Dynasore and Gefitinib, endosome disruptor Desipramine, or no treatment for 30 minutes prior to the addition of ceramide or ceramide-loaded O-GNRs.

### Statistical Analysis

Data are presented as mean±standard deviation for PrestoBlue and Caspase assays (n=6). One-way ANOVA was used to make multiple comparisons between groups, and Tukey-Kramer post hoc analysis was used to determine where significant differences occurred. Statistical analysis was performed using a 95% confidence interval (p<0.05) with Graphpad Prism.

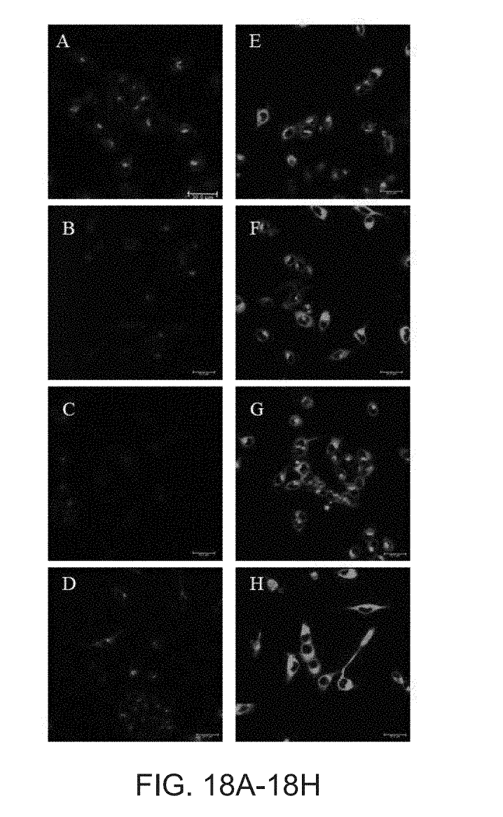

FIGS. 18A-18H are Confocal Microscopy images of HeLa cells treated with fluorescent C₁₂ NBD ceramide loaded onto O-GNRs (18A-18D) or fluorescent C₁₂ NBD ceramide only (18E-18H). Ceramide loaded onto O-GNRs (18A) shows ceramide more localized to endosomes after 2 hours than free ceramide (18E). Cells treated with Dynasore and ceramide-loaded O-GNRs (18B) and Gefitinib and ceramide-loaded O-GNRs (18C) show significantly less fluorescence than cells not treated with inhibitors of O-GNR uptake (18A).

However, cells treated with Dynasore and free ceramide (18F) and Gefitinib and free ceramide (18G) show no difference than cells treated with free ceramide alone (18E). Cells treated with free ceramide and endosome disruptor desipramine (18H) show significantly less endosome localization than cells treated with free ceramide only (18E). However, cells treated with desipramine and ceramide-loaded O-GNRs (18D) show no difference in endosome localization compared to cells treated with ceramide-loaded O-GNRs only (18A).

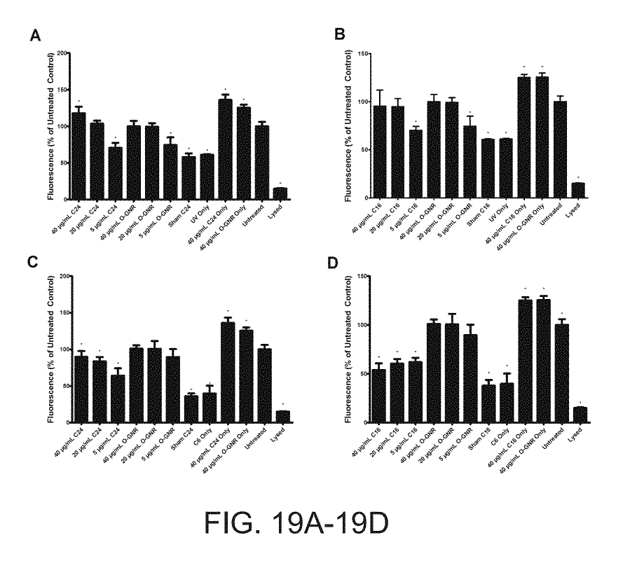

FIGS. 19A-19D are graphical illustrations of viability of HeLa cells assessed by Prestoblue. Cells were treated with either UV (19A, 19B) or C₆ ceramide (19C, 19D). Following treatment, cells were incubated with Cu ceramide (19A, 19C) and C₁₆ ceramide (19B, 19D) for 24 hours. O-GNRs without ceramide sent through the loading process were used as a control. Ceramides without nanoparticles sent through loading process were also used as controls. * indicates significant difference from “Untreated”

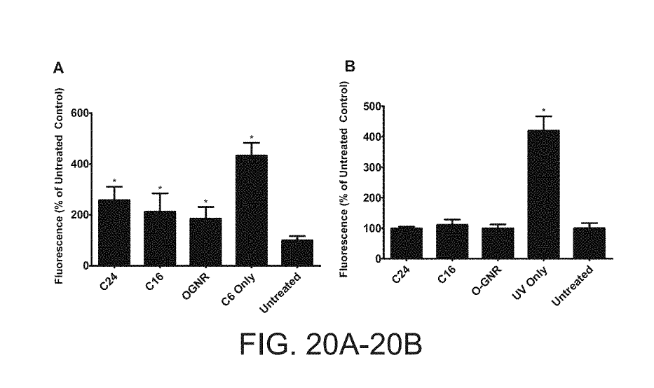

FIGS. 20A and 20B are graphical illustrations of apoptosis of HeLa cells assessed by Capase-3 activity. Cells were treated with either C₆ ceramide (20A) or UV (20B). Following treatment, cells were incubated with C₂₄ ceramide and C₁₆ ceramide for 24 hours. O-GNRs without ceramide sent through the loading process were used as a control. * indicates significant difference from “Untreated”

### Example 4

### Methods

### 1.1. Materials

C₆-Ceramide, and NBD Ceramide (C₆ N-[6-[(7-nitro-2-1,3-benzoxadiazol-4-yl)amino]hexanoyl]-D-erythro-sphingosine) were purchased from Avanti Polar Lipids (Alabaster, Ala., USA). DSPE-PEG was purchased from NOF America Corporation (White Plains, N.Y., USA). All other materials and reagents were purchased from Sigma Aldrich (St. Louis, Mo., USA) unless otherwise noted.

### 1.2. O-GNR and GNP Synthesis

O-GNRs were synthesized from multi-walled carbon nanotubes (MWCNTs) using the longitudinal unzipping method with centrifugation instead of filtration for purification. Briefly, MWCNTs (150 mg) were dispersed in 30 mL of concentrated sulfuric acid. After 3 hr, 750 mg of potassium permanganate was added and then mixture was stirred for 1 hr. The mixture was then heated to approximately 60° C. for 1 hr in an oil bath to complete the reaction. It was then allowed to cool to room temperature, and was washed with dilute hydrochloric acid. The product was isolated by flocculation using ethanol and ether, followed by centrifugation at 3000 rpm and drying overnight in a vacuum oven.

GNPs were synthesized from graphite flakes using a modified Hummers Method.

### 1.3 Atomic Force Microscopy Characterization

Dispersions of O-GNRs and GNPs in ethanol were diluted to 1 μg/mL, and 10 μL of these dispersions were drop cast onto silicon wafers (Ted Pella, Inc.). The samples were characterized using a V-shaped AFM cantilever of frequency f_{c}=145-230, tip radius less than 10 nm, and spring constant k=20-95 N/m (ACL-10, Applied NanoStructures, Inc., Mountain View, Calif., USA). Imaging was performed using a NanoSurf® EasyScan 2 FlexAFM (NanoScience Instruments, Inc., Phoenix, Ariz., USA).

1.4 Loading of Ceramide onto Carbon Nanoparticles

O-GNRs or GNPs were dispersed in 100% ethanol at a concentration of 1 mg/mL using a bath sonicator. C₆-Ceramide was dispersed in 100% ethanol at a concentration of approximately 150 μg/mL. 500 μL of the nanoparticle solution (either O-GNRs or GNPs) and 500 μL of the ceramide solution were added to a 20 mL glass scintillation vial. This mixture was bubbled with nitrogen gas and covered in parafilm to prevent oxidation of ceramide. It was then bath sonicated for 15 minutes to allow interspersion of individual nanoparticles and ceramide molecules.

To this solution, 9 mL of double-distilled water were added using a New Era NE-300 syringe pump (New Era Pump Systems Inc. Farmingdale, N.Y., USA) over a period of two hours under constant mixing. However, the water was not added at a constant rate: for the first hour, water was added at a rate of 2 mL/hr, and for the second hour, water was added at a rate of 7 mL/hr. During the first 1.5 hrs of this loading process, the ceramide/nanoparticle mixture was subjected to mild sonication using a sonic cleaner in order to prevent the clumping of nanoparticles caused by the addition of water to ethanol. In order to separate ceramide-loaded nanoparticles from unloaded ceramide, the mixture was centrifuged at 4000 rpm for at least 30 minutes in a 15 mL polypropylene conical tube. The supernatant was discarded and the nanoparticles were washed by resuspension in pure double-distilled water and repeating the centrifugation step, yielding ceramide-loaded nanoparticles.

### 1.5 Quantification of Loading Using Mass Spectrometry

Samples were prepared for mass spectrometry by first resuspending ceramide-loaded nanoparticles in 100% ethanol and bath-sonicating for 1 hr, in order to force ceramide to dissociate from the nanoparticles. The mixture was centrifuged at 13000 rpm for 1 hr in order to ensure all nanoparticles were concentrated at the bottom. 100 μL of the supernatant was extracted for mass spectrometry. 100 μL of the ceramide stock solution used for loading was also analyzed as a basis of comparison to calculate loading efficiency. Samples were analyzed using a Fisons MD800 Gas Chromatography Mass Spectrometer. The signals generated from samples were correlated to a standard curve in order to calculate the exact concentration of ceramide.

### 1.6 Cell Culture

HeLa cells were obtained from ATCC (Manassas, Va., USA). Cells were grown in Dulbecco's Modified Eagle Medium (DMEM) supplemented with 10% fetal bovine serum and 1% penicillin-streptomycin. Cells were grown at 37° C. in a humidified atmosphere of 5% carbon dioxide.

### 1.7 PrestoBlue Viability Assay

HeLa cells were seeded in flat-bottomed 96 well plates at a density of 5000 cells/well and were allowed to adhere overnight. Ceramide-loaded nanoparticles (O-GNRs or GNPs) were added to each well at various concentrations ranging from 10-100 μg/mL of nanoparticles. In order to ensure that any observed decrease in viability compared to untreated cells could not be attributed to the loading process, the loading process was repeated with only nanoparticles and incubated them with HeLa cells at the highest concentration used (100 μg/mL). After 24 hours, the media was aspirated and each well was rinsed twice with PBS. A mixture of 10 μL of Presto Blue Viability Reagent (Life Technologies, Grand Island, N.Y., USA) and 90 μL of DMEM with 10% FBS and 1% P/S were added to each well and placed back in the incubator. After 2 hours, the plates were removed and the fluorescence intensity of each well was measured using a Molecular Devices SpectraMax M2e (Sunnyvale, Calif., USA) with an excitation wavelength of 560 nm and an emission wavelength of 590 nm. Lysed cells were used as a positive control, and untreated cells were used as a negative control. The cell viability is expressed as a percent of the lysis control with the formula:

((Iₜₑₛₜ−I_{blank})/(I_{control}−I_{blank})×100%)−100%

where Iₜₑₛₜ is the fluorescence intensity of cells exposed to ceramide-loaded nanoparticles, I_{control} is the fluorescence intensity of lysed cells, and I_{blank} is the fluorescence intensity from empty wells.

In a separate experiment, the efficacy of ceramide-loaded O-GNRs were compared to C₆ ceramide without nanoparticles placed directly in the media. A stock solution of C₆ ceramide in 100% ethanol or ceramide-loaded O-GNRs in water were diluted in water to concentrations ranging from 2-10 μg/mL of C₆. Ethanol diluted in water or O-GNRs alone served as a control for the delivery vehicle. Viability was assessed with PrestoBlue using identical methods as described above.

### 1.8 Hoechst Stain for Apoptosis

HeLa cells were seeded at a density of 7.5×10⁴ cells/mL in 6 well plates and allowed to attach overnight. Ceramide-loaded O-GNRs were added at a concentration of 40 μg/mL for 24 hrs, with non-loaded O-GNRs serving as a control. After 24 hrs, the media was removed and saved. Cells were then rinsed with PBS (which was also saved) and then trypsinized. Detached cells were added together with the removed DMEM and PBS, and centrifuged at 1000 rpm for 5 minutes. The pellet was resuspended in 10% paraformaldehyde until needed. To visualize apoptotic nuclei, the cells were centrifuged and resuspended in a 1% solution of bisBenzimide H 33342 trihydrochloride for 10 minutes. Cells were imaged using a Zeiss Axio Imager M2 (Thornwood, N.Y., USA) with an excitation wavelength of ˜350 nm and emission wavelength of ˜460 nm.

### 1.9 Live-Cell Confocal Microscopy of HeLa Cells

In order to visualize the uptake of ceramide-loaded nanoparticles into cells, we loaded O-GNRs with C₆ ceramide conjugated to 4-Chloro-7-nitrobenzofurazan (NBD-ceramide). HeLa cells were seeded in glass-inset confocal dishes at a density of 7.5×10⁴ cells/mL with 2 mL per plate and allowed to adhere overnight. The following day, cells were placed in a LiveCell™ stage top incubation platform (Pathology Devices Inc., Westminster, Md., USA) and NBD-ceramide-loaded O-GNRs were added to these plates at a concentration of 40 μg/mL. As a control, cells were also incubated with an equivalent amount of NBD-ceramide in ethanol in a separate experiment. The cells were then imaged using a Leica TCS SP8 Laser Scanning Confocal Microscope System (Buffalo Grove, Ill., USA) over a period of one hour at two minute intervals.

### 1.10 TEM Visualization of Uptake of C6-Loaded O-GNRs

HeLa cells were grown on ACLAR® film (Electron Microscopy Sciences, Hatfield, Pa.) in 6 well plates at a density of 20000 cells per well and allowed to settle overnight. The following day, cells were exposed to C₆-loaded O-GNRs for one hour. The cells were subsequently fixed with 2.5% glutaraldehyde (Electron Microscopy Sciences, Hatfield, Pa.) in 0.1 M PBS for 15 minutes at room temperature. The films were then placed in 2% osmium tetroxide in 0.1 M PBS, dehydrated through graded ethanol washes, and embedded using durcupan resin. The embedded specimens were screened for areas with high cell densities, and these areas were cut into 80 nm sections using an Ultracut E microtome (Reichert-Jung, Cambridge, UK), and placed on formvar-coated copper grids. Sections were imaged using a Tecnai Bio Twin G transmission electron microscope (FEI, Hillsboro, Oreg.), at 80 kV. Images were acquired using an XR-60 CCD digital camera system (AMT, Woburn, Mass.).

### 2. Statistical Analysis

Data are presented as mean±standard deviation for PrestoBlue assays (n=6). One-way ANOVA was used to make multiple comparisons between groups, and Tukey-Kramer post hoc analysis was used to determine where significant differences occurred. Statistical analysis was performed using a 95% confidence interval (p<0.05) with Graphpad Prism.

### 3. Results

### 3.1 AFM Characterization

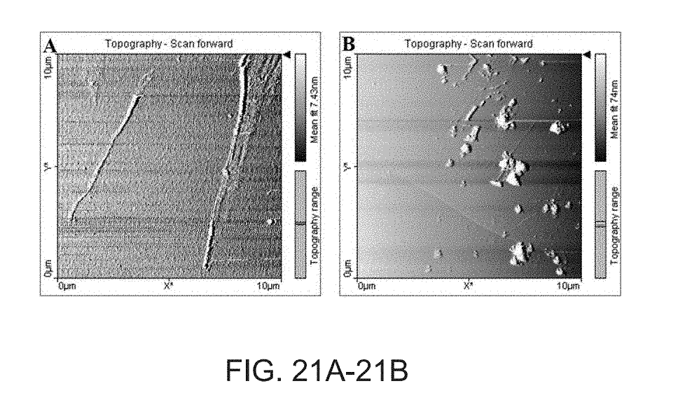

O-GNRs characterized with AFM had a size range of mainly 50-2500 nm with a high aspect ratio characteristic of carbon nanotubes. Several particles were as long as 8000 nm (FIG. 21A). The thickness of these particles ranged from 4-10 nm, indicating that they were several-layers thick. The observed dimensions for GNPs were much smaller in range, mainly between 20 and 500 nm with slightly irregular shapes and aspect ratios closer to one (FIG. 21B). The observed thickness of GNPs were comparable to O-GNRs, with thicknesses ranging between 2 and 12 nm. 3.2 C₆ Ceramide Loading onto Graphene Nanoparticles

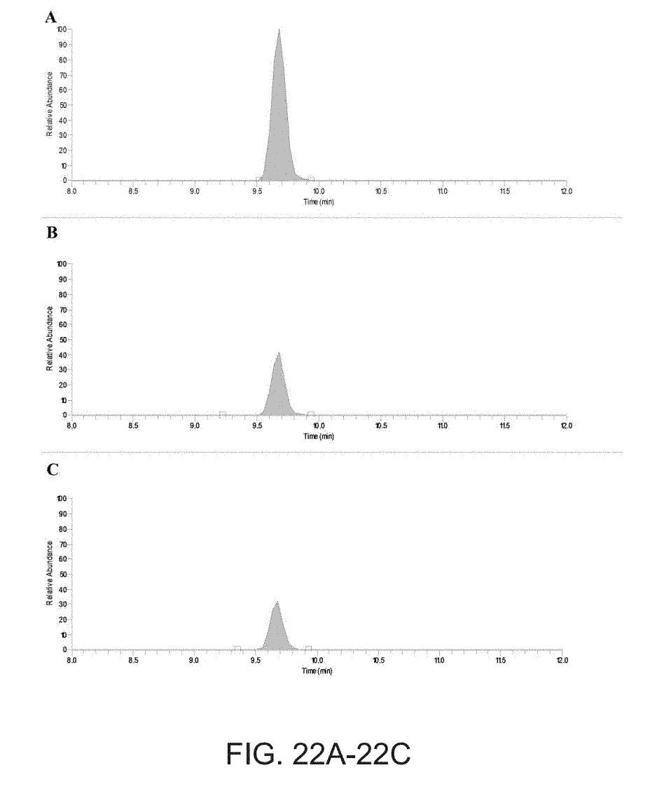

FIGS. 22A-22C display the raw data readout of relative abundances of C₆ ceramide as determined by mass spectrometry. The readout is standardized with the stock solution as 100 (FIG. 22A) to which the relative abundances of C₆ ceramide loaded onto O-GNRs (FIG. 22B) and GNPs (FIG. 22C). Although these raw data show the relative differences of O-GNR and GNP C₆ ceramide loading, the actual concentrations of ceramide found need to be determined from a C6 standard curve calculated with known concentrations of C₆ ceramide. Using this standard curve, the loading efficiencies for O-GNRs and GNPs was determined to be approximately 57% and 51.5%, respectively. On a mass per mass basis, this represents at least a 50% increase in incorporation efficiency compared to C₆ ceramide incorporation into liposomes.

### 3.3 PrestoBlue Viability Assay

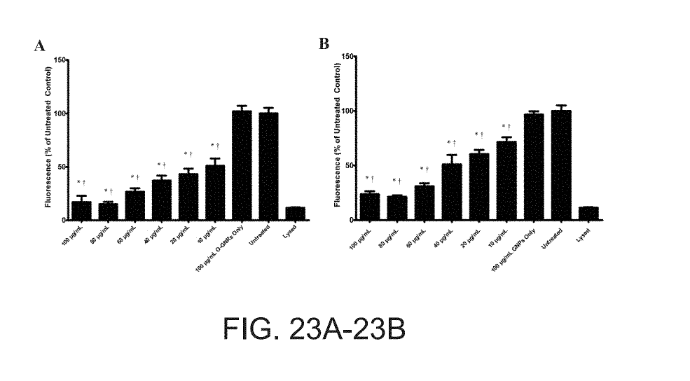

After determining that the loading method was loading high amounts of drug onto nanoparticles, the next step was to determine if the delivery system was capable of toxicity to HeLa cells. PrestoBlue is a resazurin dye that can be reduced by living cells to a form that fluoresces intensely at 590 nm when excited at 560 nm. Thus, the fluorescence measured from this dye is a good indicator of cell viability. HeLa cells were incubated with concentrations of O-GNRs and GNPs loaded with C₆-ceramide ranging from 10-100 μg/mL of nanoparticles. After 2 hours of incubation with PrestoBlue, it was determined that C₆ ceramide-loaded O-GNRs and GNPs were significantly toxic to all concentrations tested (FIGS. 23A, 23B). For O-GNRs, 100 g/mL reduced cell viability so much that these cells were only 5.51% more viable than lysed cells. Even at the lowest concentration tested (10 μg/mL), these cells were only 39% more viable than lysed cells (FIG. 23A). For GNPs, at the highest concentration of 100 μg/mL tested, cells were only 12.41% more viable than lysed cells, and 60.12% more viable than lysed cells at the lowest concentration tested.

Since carbon nanoparticles are often combined with agents that increase their dispersibility in aqueous solution, it was determined if a dispersion agent would have any negative effect on the toxicity of the C₆-ceramide delivery system. Thus, after loading and washing the C₆-ceramide-loaded nanoparticles, they were resuspended in a 1.2 mg/mL solution of DSPE-PEG—which has previously been used with graphene nanoparticles—and the PrestoBlue viability assay was repeated.

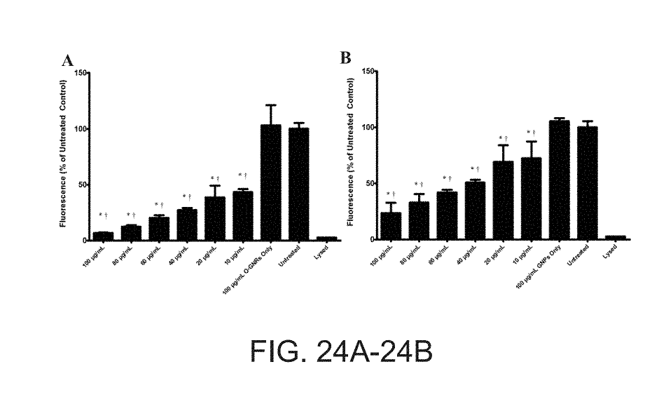

FIGS. 24A and 24B illustrate that resuspension in DSPE-PEG did not have a negative impact on the toxicities of C₆-ceramide-loaded O-GNRs and C6-loaded GNPs, respectively. HeLa cells incubated with C6-loaded O-GNRs were only 4.01% and 40.5% more viable than lysed cells at concentrations of 100 μg/mL and 10 μg/mL, respectively, which was very significant compared to the viability of untreated cells, and cells treated with O-GNRs only (FIG. 24A). This toxicity was relatively the same compared to cells incubated with C6-loaded O-GNRs not resuspended in DSPE-PEG (FIG. 23A). There was a similar result in toxicity for cells incubated with C6-loaded GNPs in DSPE-PEG, with a reduction in viability of 20.82% and 69.61% at concentrations of 100 μg/mL and 10 μg/mL, respectively (FIG. 24B).

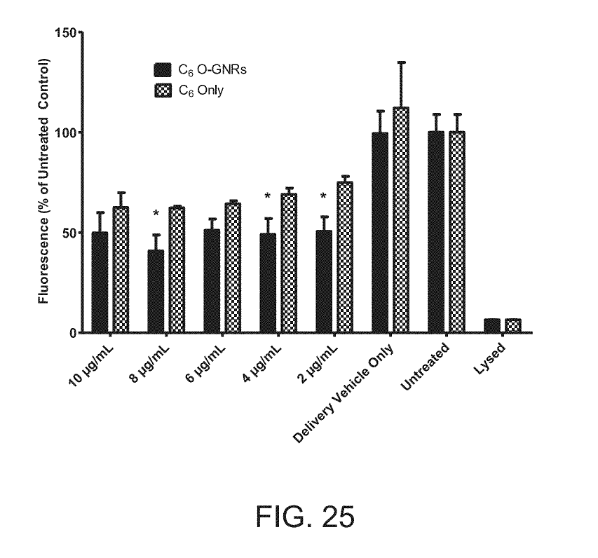

O-GNRs were significantly more toxic than C₆ in ethanol alone at concentrations of 8, 4, and 2 μg/mL (FIG. 5). At the highest level of toxicity (8 μg/mL), HeLa cell viability was 34.34% greater than lysed cells for C₆ O-GNRs, while HeLa cell viability was 55.8% greater than lysed for C₆ alone (FIG. 25).

### 3.4 Hoechst Stain for Apoptosis

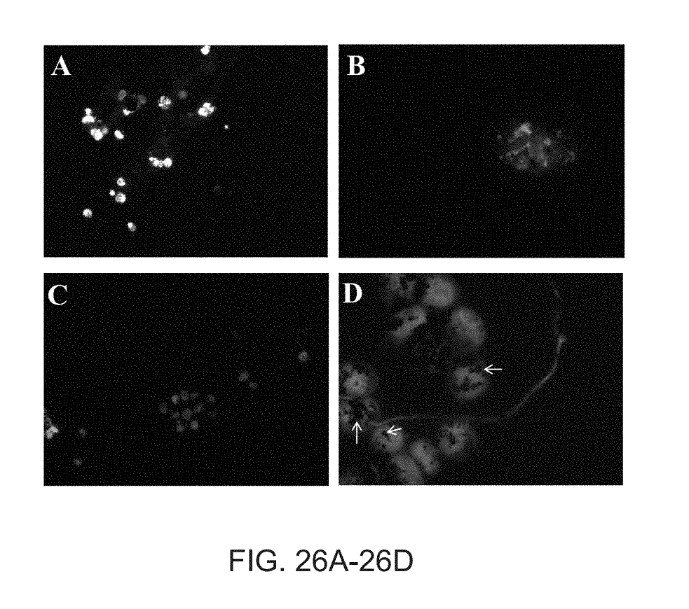

Since the most commonly reported mode of cell death induced by ceramide C₆-ceramide is apoptosis, it was determined whether the cell death observed was apoptotic in nature. Hoeschst stain binds to DNA, and under UV light, apoptotic cells appear much brighter than their non-apoptotic counterparts as a result of condensed chromatin and nuclear fragmentation. After exposure to 40 μg/mL O-GNRs loaded with C₆ ceramide, HeLa cells stained with bisbenzimide displayed bright nuclei (FIG. 26A) and nuclear fragmentation (FIG. 26B) characteristic of apoptosis. By contrast, HeLa cells exposed to 40 μg/mL O-GNRs did not exhibit any signs of apoptosis (FIG. 26C) despite the presence of high levels of O-GNRs on the surface of cells (FIG. 26D). This indicates that the C₆-ceramide-loaded O-GNRs, and not unloaded O-GNRs, were inducing significant levels of apoptosis.

### 3.5 Live-Cell Confocal Microscopy of Fluorescent Ceramide

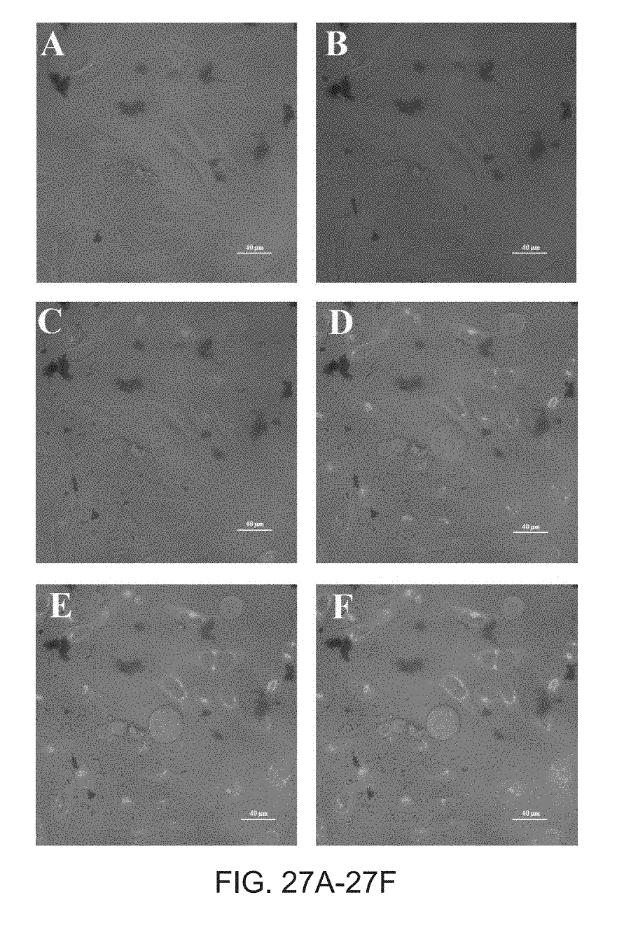

FIGS. 27A-27F show time-lapsed images (12 minute intervals) of live HeLa cells incubated with 40 μg/mL NBD ceramide-loaded O-GNRs. By using the fluorescently-tagged NBD ceramide in the delivery system and in combination with live cell imaging, it was determined which parts of the cells ceramide is localizing. Immediately after the addition of NBD ceramide-loaded O-GNRs, there was no visible fluorescence (FIG. 27A) and minimal fluorescence after 12 minutes (FIG. 27B). There is some green NBD fluorescence present after 24 minutes, possibly due to some NBD-ceramide on the O-GNRs crystallizing and falling off (FIG. 27C). Between 24 and 36 minutes, there is a sudden large increase in fluorescence, with some substructures of cells being visibly highlighted by NBD ceramide (FIG. 27D). This sudden increase in fluorescence is possibly related to the uptake of O-GNRs, which are uptaken into HeLa cells in as little as 15 minutes. The intensity of this fluorescence continued to increase after 48 and 60 minutes, indicating that the entry of NBD ceramide into HeLa cells did not diminish over time and possibly continued beyond the one hour time frame the study (FIGS. 27E and 27F).

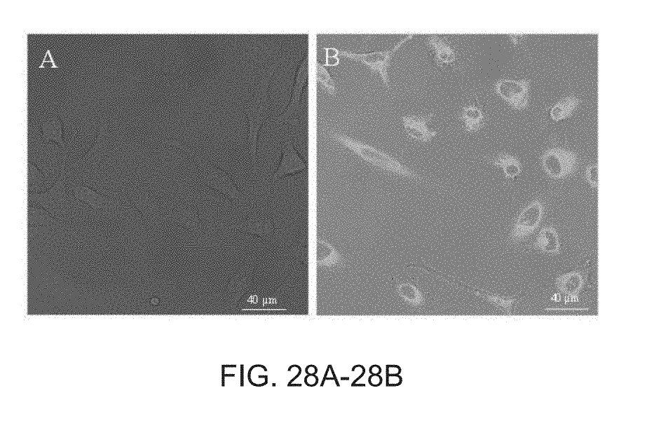

In order to see if there was a difference in the uptake between our C₆-loaded O-GNRs and C₆ alone, the same experiment was performed using an equal concentration of NBD-ceramide in ethanol alone. HeLa cells incubated with NBD ceramide alone demonstrated a rapid uptake into HeLa cells. Prior to addition of NBD ceramide, there was no visible fluorescence (FIG. 28A). However, between the time that the NBD ceramide was added and images were taken, there was already a large amount of observable fluorescence (FIG. 28B). This extremely rapid uptake profile would suggest that NBD ceramide loaded onto O-GNRs is not merely dissociating from the nanoparticles in proximity to cells, but remaining bound to the O-GNRs and being released as the nanoparticles are uptaken by the cells.

3.6 TEM of C6-Loaded O-GNRs Uptake into HeLa Cells

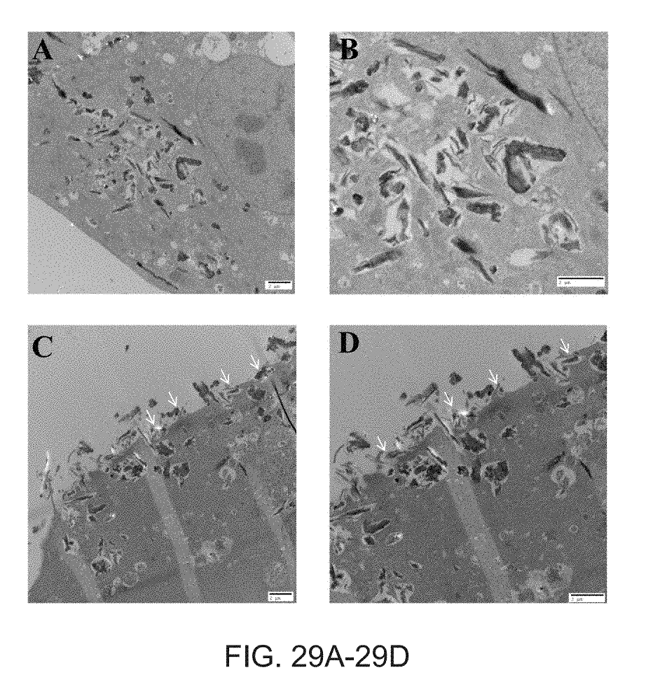

Although there were large aggregates of O-GNRs visible under confocal microscopy (FIGS. 27A-27F), the much smaller particles that would be actually be entering cells and delivering ceramide were not visible from this magnification. And while uptake of these particles into cells has been shown, it was not clear if the loading of ceramide onto O-GNRs were altering the route of entry into cells, or if these nanoribbons were entering cells at all. FIGS. 29A-29D are TEM images of HeLa cells incubated with C6-loaded nanoribbons for one hour. There was a high degree of uptake of O-GNRs, with many O-GNRs of different shapes and sizes contained within vacuoles visible (FIGS. 29A and 29B). Further examination of other HeLa cells in the samples revealed the uptake process in progress, with the outward blebbing of the cell membrane to surround the O-GNRs clearly visible (FIGS. 29C and 29D, white arrows). This uptake modality of O-GNRs is similar to the macropinocytotic-like process previously observed in HeLa cells_ENREF_13. Thus, it does not seem that loading ceramide onto O-GNRs affects their entry into HeLa cells.

### 4. Discussion

Despite its potent pro-apoptotic effects, there is currently no clinically-approved in vivo method of delivering ceramide. This disclosure describes a method for loading the short-chain C₆ ceramide onto graphene nanoparticles.

The disclosed method does not rely on any specific formulation or manipulation of lipids themselves, but achieves an interface between graphene and ceramide by bulk hydrophobicity. And although ceramide has been tested, the theoretical basis for this loading method would allow for any lipid or combination of lipids to be loaded onto graphene nanoparticles.

O-GNRs were able to load more C₆ ceramide than GNPs, representing a difference in loading efficiencies of approximately 6.5% (FIGS. 22A-22C).

This difference in loading is also reflected in viability as determined by PrestoBlue, where C₆-ceramide-loaded O-GNRs generally reduced HeLa cell viability more than C₆-ceramide-loaded GNPs. Although the toxicity of both systems was relatively the same at highest dose of 100 μg/mL, O-GNRs maintained higher toxicity all the way to the lowest dose of 10 μg/mL (FIGS. 23A & 23B). This same trend continued even when C₆-ceramide-loaded nanoparticles were resuspended in DSPE-PEG (FIGS. 24A & 24B). Although this difference in cytotoxicity between the two types of nanoparticles could be partly due to the differences in loading, this could also possibly due to the selective uptake property of O-GNRs. The uptake of O-GNRs observed via TEM showed a significant number of visible nanoparticles taken up by HeLa cells (FIGS. 29A-29D).

Although exogenous ceramide is not normally soluble in aqueous solution, it can be delivered in vitro to cells using an organic solvent such as ethanol (albeit with low efficiency). Upon delivery to the cell, ceramide tends to localize mostly in endosomes, particularly lysosomes. Using live-cell confocal imaging of NBD-ceramide uptake into HeLa cells, significant fluorescence in HeLa cells was observed in as little as 24 minutes after delivery, and a large increase in fluorescence between the 24 minute and 36 minute time points (FIGS. 27C and 27D) was observed.

However, while the fluorescence at 24 minutes is more generalized and even throughout parts of the cells, at 36 minutes there are distinct cellular substructures exhibiting fluorescence (FIG. 27D). One possible explanation for this phenomenon is related to the macropinocytotic-like mechanism by which O-GNRs enter cells. This specific increase in fluorescence may correspond to a bulk uptake of O-GNRs loaded with NBD-ceramide, with a subsequent bulk release of NBD-ceramide from O-GNRs inside the macropinosome during its relatively rapid (2-4 minutes) maturation process.

A method for the loading of ceramide onto oxidized graphene nanoparticles is disclosed, with these ceramide-loaded nanoparticles demonstrating induced apoptosis in HeLa cells and uptake of occurring rapidly.

### Example 5

The present disclosure can include myelin basic protein (“MBP”) and variants thereof (including isoforms), and certain fragments of MBP, to interfere with fibrillization of a fibrillizing peptide, Aβ in particular. This MBP can be delivered to a cell of a subject by being loaded onto carbon nanoparticles, such as oxidized graphene nanoribbons (O-GNRs). These loaded O-GNRs can be delivered to a subject in any suitable way, including intravenously and through targeted injections. Due to the size of the loaded O-GNRs they can pass through the blood brain barrier and the MBP loaded onto the O-GNRs can be delivered to brain cells.

MBP refers herein to a protein involved in the myelination of nerves in mammalian subjects. In mammals, various forms of MBP exist which are produced by the alternative splicing of a single gene; these forms differ by the presence or the absence of short (10 to 20 residues) peptides in various internal locations in the sequence. The major form of MBP is generally a protein of about 18.5 Kd (170 residues). MBP is the target of many post-translational modifications: it is N-terminally acetylated, methylated on an arginine residue, phosphorylated by various serine/threonine protein-kinases, and deamidated on some glutamine residues.

MBP is encoded by a member of a large family of developmentally regulated genes called the Golli complex (genes of the oligodendrocyte lineage) (Campagnoni et al., J. Biol. Chem. 268:4930-4938, 1993, Pribyl et al., Proc. Natl. Acad. Sci. U.S.A. 90:10695-10699, 1993, Givogri et al., J. Neurosci. Res. 59:153-159, 2000). Members of this family are involved in the formation and maintenance of myelin sheaths. However, Golli proteins are also found in fetal spinal cord, thymus, spleen, and in cells derived from the immune system (Givogri et al., J. Neurosci. Res. 59:153-159, 2000), as well as in neurons (Tosic et al., Glia 37:219-228, 2002), Pribyl et al., J. Comp. Neurol. 374:342-353, 1996). The Golli locus contains two distinct start sites under independent regulation and consists of 11 exons that can be alternatively spliced to form the various Golli-MBPs. Included are seven exons that encode the proteins (Pribyl et al., Proc. Natl. Acad. Sci. U.S.A. 90:10695-10699, 199). The major species are 21.5, 20.2, 18.5, and 17.2 kDa (Roth et al., J. Neurosci. Res. 17:321-328, 1987). The 21.5-, 20.2-, and 17.2-kDa isoforms are found in fetal and developing brains. In adults, the 18.5- and 17.2-kDa isoforms are predominant (Baumann and Pham-Dinh, Physiol. Rev. 81:871-927, 2001).

MBP includes four isoforms having the following sequence identifiers:

TABLE 1
Nucleotide Sequence for Isoform 1 of Homo sapiens MBP (SEQ ID NO: 1)
Isoform 1: (NM_001025081.1)

| | |
|---|---|
| 1 | ggacaacacc ttcaaagaca ggccctctga gtccgacgag ctccagacca tccaagaaga |
| 61 | cagtgcagcc acctccgaga gcctggatgt gatggcgtca cagaagagac cctcccagag |
| 121 | gcacggatcc aagtacctgg ccacagcaag taccatggac catgccaggc atggcttcct |
| 181 | cccaaggcac agagacacgg gcatccttga ctccatcggg cgcttctttg gcggtgacag |
| 241 | gagtgcaccc aagcgaggct ctggcaaggt accctggcta aaaccggacc ggagccctct |
| 301 | gccctctcat gcccgcagcc agcctgggct gtgcaacatg tacaaggact cacaccaccc |
| 361 | gacaagaact gctcactacg gctccctgcc ccagaagtca cacggccgga cccaagatga |
| 421 | aaaccccgta gtccacttct tcaagaacat tgtgacgcct cgcacaccac ccccgtcgca |
| 481 | gggaaagggg agaggactgt ccctgagcag atttagctgg ggggccgaag gccagagacc |
| 541 | aggatttggc tacggaggca gagcgtccga ctataaatcg gctcacaagg gattcaaggg |
| 601 | agtcgatgcc cagggcacgc tttccaaaat ttttaagctg ggaggaagag atagtcgctc |
| 661 | tggatcaccc atggctagac gctgaaaacc cacctggttc cggaatcctg tcctcagctt |
| 721 | cttaatataa ctgccttaaa actttaatcc cacttgcccc tgttacctaa ttagagcaga |
| 781 | tgacccctcc cctaatgcct gcggagttgt gcacgtagta gggtcaggcc acggcagcct |
| 841 | accggcaatt tccggccaac agttaaatga gaacatgaaa acagaaaacg gttaaaactg |
| 901 | tcccattctg tgtgaagatc acgttccttc ccccgcaatg tgcccccaga cgcacgtggg |
| 961 | tcttcagggg gccaggtgca cagacgtccc tccacgttca cccctccacc cttggacttt |
| 1021 | cttttcgccg tggctgcggc acccttgcgc ttttgctggt cactgccatg gaggcacaca |
| 1081 | gctgcagaga cagagaggac gtgggcggca gagaggactg ttgacatcca agcttccttt |
| 1141 | gttttttttt cctgtccttc tctcacctcc taaagtagac ttcatttttc ctaacaggat |
| 1201 | tagacagtca aggagtggct tactacatgt gggagctttt agtatgtgac atgcgggctg |
| 1261 | gacagctgtt agagtccaac gtggggcagc acagagaggg agccacctcc ccagaccgtg |
| 1321 | gctgcccaca caccccaatt agctgaattc gcgtgtggca gagggaggaa aaggaggcaa |
| 1381 | acgtgggctg ggcaatggcc tcacatagga aacagggtct tcctggagat ttggtgatgg |
| 1441 | agatgtcaag caggtggcct ctggacgtca ccgttaccct gcatggtggc cccagagcag |
| 1501 | cctctatgaa caacctcgtt tccaaaccac agcccacagc cggagagtcc aggaagactt |
| 1561 | gcgcactcag agcagaaggg taggagtcct ctagacagcc tcgcagccgc gccagtcgcc |
| 1621 | catagacact ggctgtgacc gggcgtgctg gcagcggcag tgcacagtgg ccagcactaa |
| 1681 | ccctccctaa gaagataacc ggctcattca cttcctccca gaagacgcat agtagcgagt |
| 1741 | agacacaggc gtgcacctgc tcccgaatta ctcaccgaga cacacgggct gagcagacgg |
| 1801 | ccccgtagat ggagacaaag agctcttctg accatatcct tcttaacacc cgctggcatc |
| 1861 | tcctttcgcg cctccctccc taacctactg acccaccttt tgattttagc gcacctgtga |
| 1921 | ttgataggcc ttccaaagag tcccacgctg gcatcaccct ccccgaggac ggagatgagg |
| 1981 | agtagtcagc gtgatgccaa aacgcgtctt cttaatccaa ttctaattct gaatgtttcg |
| 2041 | tgtgggctta ataccatgtc tattaatata tagcctcgat gatgagagag ttacaaagaa |
| 2101 | caaaactcca gacacaaacc tccaaatttt tcagcagaag cactctgcgt cgctgagctg |
| 2161 | aggtcggctc tgcgatccat acgtggccgc acccacacag cacgtgctgt gacgatggct |
| 2221 | gaacggaaag tgtacactgt tcctgaatat tgaaataaaa caataaactt ttaatggtaa |
| 2281 | aaaaaaaaaa aaaaaaaaaa |

TABLE 2
Nucleotide Sequence for Isoform 2 of Homo sapiens MBP (SEQ ID NO: 2)
Isoform 2: (NM_002385.2)

| | |
|---|---|
| 1 | ggacaacacc ttcaaagaca ggccctctga gtccgacgag ctccagacca tccaagaaga |
| 61 | cagtgcagcc acctccgaga gcctggatgt gatggcgtca cagaagagac cctcccagag |
| 121 | gcacggatcc aagtacctgg ccacagcaag taccatggac catgccaggc ataacttcct |
| 181 | cccaagacac agagacacgg gcatccttga ctccatcggg cgcttctttg gcggtgacag |
| 241 | gggtacgccc aaacggggct ctggcaagat accctgacta aagccgggcc ggagccctct |
| 301 | gccctctcat acccgcaacc agcctgggct gtgcaacatg tacaaggact cacaccaccc |
| 361 | ggcaagaact gctcactaca gctccctacc ccagaagtca cacggccgga cccaagataa |
| 421 | aaaccccgta gtccacttct tcaagaacat tgtgacgcct cgcacaccac ccccgtcgca |
| 481 | gggaaagggg gccgaaggcc agagaccagg atttggctac ggaggcagag cgtccgacta |
| 541 | taaatcggct cacaagggat tcaagggagt cgatgcccag ggcacgcttt ccaaaatttt |
| 601 | taaactgaaa agaagagata gtcgctctgg atcacccatg gctagacgct gaaaacccac |
| 661 | ctggttccgg aatcctgtcc tcagcttctt aatataactg ccttaaaact ttaatcccac |
| 721 | ttgcccctgt tacctaatta aagcagatga cccctcccct aatgcctgcg gagttgtgca |
| 781 | cgtagtaggg tcaggccacg gcagcctacc ggcaatttcc ggccaacagt taaatgagaa |
| 841 | catgaaaaca gaaaacggtt aaaactgtcc ctttctgtgt gaagatcacg ttccttcccc |
| 901 | cgcaatgtgc ccccagacgc acgtgggtct tcagggggcc aggtgcacag acgtccctcc |
| 961 | acgttcaccc ctccaccctt ggactttctt ttcgccgtgg ctgcggcacc cttgcgcttt |
| 1021 | tgctggtcac tgccatggag gcacacagct gcagagacag agaggacgtg ggcggcagag |
| 1081 | aggactgttg acatccaagc ttcctttgtt tttttttcct gtccttctct cacctcctaa |
| 1141 | agtagacttc atttttccta acaggattag acagtcaagg agtggcttac tacatgtggg |
| 1201 | agcttttggt atgtgacatg cgggctgggc agctgttaga gtccaacgtg gggcagcaca |
| 1261 | gagagggggc cacctcccca ggccgtggct gcccacacac cccaattagc tgaattcgcg |
| 1321 | tgtggcagag ggaggaaaag gaggcaaacg tgggctgggc aatggcctca cataggaaac |
| 1381 | agggtcttcc tggagatttg gtgatggaga tgtcaagcag gtggcctctg gacgtcaccg |
| 1441 | ttgccctgca tggtggcccc agagcagcct ctatgaacaa cctcgtttcc aaaccacagc |
| 1501 | ccacagccgg agagtccagg aagacttgcg cactcagagc agaagggtag gagtcctcta |
| 1561 | gacagcctcg cagccgcacc agtcgcccat agacactggc tgtgaccggg cgtgctggca |
| 1621 | acggcagtgc acagtggcca gcactaaccc tccctgagaa aataaccggc tcattcactt |
| 1681 | cctcccagaa gacgcgtggt agcgagtagg cacaggcgtg cacctgctcc cgaattactc |
| 1741 | accgagacac acgggctgaa cagacggccc cgtggatgga gacaaagagc tcttctgacc |
| 1801 | atatccttct taacacccac tggcatctcc tttcgcgcct ccctccctaa cctactaacc |
| 1861 | caccttttga ttttagcgca cctgtgattg ataggccttc caaagagtcc cacgctggca |
| 1921 | tcaccctccc cgaggacgga gatgaggagt agtcagcgtg atgccaaaac gcgtcttctt |
| 1981 | aatccaattc taattctgaa tgtttcgtgt gggcttaata ccatgtctat taatatatag |
| 2041 | cctcgatgat gagagaatta caaagaacaa aactccagac acaaacctcc aaatttttca |
| 2101 | gcagaagcac tctacgtcgc tgagctaagg tcggctctgc gatccatacg tggccgcacc |
| 2161 | cacacagcac gtgctgtgac gataactgaa cggaaagtgt acactgttcc tgaatattaa |
| 2221 | aataaaacaa taaactttta atggtaaaaa aaaaaaaaaa aaaaaaa |

TABLE 3
Nucleotide Sequence for Isoform 3 of Homo sapiens MBP (SEQ ID NO: 3)
Isoform 3: (NM_001025090.1)

| | |
|---|---|
| 1 | ggacaacacc ttcaaagaca ggccctctga gtccgacgag ctccagacca tccaagaaga |
| 61 | cagtgcagcc acctccgaga gcctggatgt gatggcgtca cagaagagac cctcccagag |
| 121 | gcacggatcc aagtacctgg ccacagcaag taccatggac catgccaggc atggcttcct |
| 181 | cccaaggcac agagacacgg gcatccttga ctccatcggg cgcttctttg gcggtgacag |
| 241 | gggtgcgccc aagcggggct ctggcaagga ctcacaccac ccggcaagaa ctgctcacta |
| 301 | cggctccctg ccccagaagt cacacggccg gacccaagat gaaaaccccg tagtccactt |
| 361 | cttcaagaac attgtgacgc ctcgcacacc acccccgtcg cagggaaagg ggagaggact |
| 421 | gtccctgagc agatttagct ggggggccga aggccagaga ccaggatttg gctacggagg |
| 481 | cagagcgtcc gactataaat cggctcacaa gggattcaag ggagtcgatg cccagggcac |
| 541 | gctttccaaa atttttaagc tggaaggaag agatagtcgc tctggatcac ccatggctag |
| 601 | acgctgaaaa cccacctggt tccggaatcc tgtcctcagc ttcttaatat aactgcctta |
| 661 | aaactttaat cccacttgcc cctattacct aattagagca gatgacccct cccctaatgc |
| 721 | ctgcggagtt atgcacgtag tagggtcagg ccacggcagc ctaccggcaa tttccgacca |
| 781 | acagttaaat gagaacataa aaacagaaaa cggttaaaac tgtccctttc tgtgtaaaga |
| 841 | tcacgttcct tcccccgcaa tgtgccccca gacgcacgtg ggtcttcagg gggccaggtg |
| 901 | cacagacgtc cctccacgtt cacccctcca cccttggact ttcttttcgc cgtggctgcg |
| 961 | gcacccttgc gcttttgctg gtcactgcca tggaggcaca cagctgcaga gacagagagg |
| 1021 | acgtgggcgg cagagaggac tgttgacatc caagettect ttattttttt ttcctgtcct |
| 1081 | tctctcacct cctaaagtaa acttcatttt tcctaacaaa attagacagt caaggaatgg |
| 1141 | cttactacat gtgggagctt ttggtatatg acatgcgggc tgggcagctg ttaaagtcca |
| 1201 | acgtggggca gcacagagag ggggccacct ccccaggccg tggctgccca cacaccccaa |
| 1261 | ttagctgaat tcgcgtgtgg cagagggagg aaaaggaggc aaacgtgggc tgggcaatgg |
| 1321 | cctcacatag gaaacagggt ettectggag atttggtgat ggagatgtca agcaggtggc |
| 1381 | ctctggacgt caccgttgcc ctgcatggtg gccccagagc agcctctatg aacaacctcg |
| 1441 | tttccaaacc acagcccaca gccggagagt ccaggaagac ttgcgcactc agagcagaag |
| 1501 | ggtaggagtc ctctagacag cctcgcagcc gcgccagtcg cccatagaca ctggctgtga |
| 1561 | ccgggcgtgc tggcagcggc agtgcacagt ggccagcact aaccctcect gagaagataa |
| 1621 | ccggctcatt cacttcctcc cagaagacgc gtggtagcga gtaggcacag gcgtgcacct |
| 1681 | gacccgaat tactcaccga gacacacggg ctgagcagac ggccccgtgg atggagacaa |
| 1741 | agagctcttc tgaccatatc cttcttaaca cccgctggca tctcctttcg cgcctccctc |
| 1801 | cctaacctac tgacccacct tttgatttta gcgcacctgt gattgatagg ccttccaaag |
| 1861 | agtcccacgc tggcatcacc ctccccgagg acggagatga ggagtagtca gcgtgatgcc |
| 1921 | aaaacgcgtc ttcttaatcc aattctaatt ctgaatgttt cgtgtgggct taataccatg |
| 1981 | tctattaata tatagcctcg atgatgagaa aattacaaag aacaaaactc cagacacaaa |
| 2041 | cctccaaatt tttcagcaga agcactctgc gtcgctgagc tgaggtcggc tctgcgatcc |
| 2101 | atacatggcc gcacccacac agcacgtact gtgacgatgg ctaaacggaa aatgtacact |
| 2161 | attcctaaat attgaaataa aacaataaac ttttaatggt aaaaaaaaaa aaaaaaaaaa |
| 2221 | aa |

TABLE 4
Nucleotide Sequence for Isoform 4 of Homo sapiens MBP (SEQ ID NO: 4)
Isoform 4: (NM_001025092.1)

| | |
|---|---|
| 1 | ggacaacacc ttcaaagaca ggccctctga gtccgacgag ctccagacca tccaagaaga |
| 61 | cagtgcagcc acctccgaga gcctggatgt gatggcgtca cagaagagac cctcccagag |
| 121 | gcacggatcc aagtacctgg ccacagcaaa taccatggac catgccaggc atggcttcct |
| 181 | cccaaggcac agagacacgg gcatccttga ctccatcggg cgcttctttg gcggtgacag |
| 241 | gggtgcgccc aagcggggct ctggcaagga ctcacaccac ccggcaagaa ctgctcacta |
| 301 | cggctccctg ccccagaagt cacacggccg gacccaagat gaaaaccccg tagtccactt |
| 361 | cttcaagaac attgtgacgc ctcgcacacc acccccgtcg cagggaaagg gggccgaagg |
| 421 | ccagagacca ggatttggct acggaggcag agcgtccgac tataaatcgg ctcacaaggg |
| 481 | attcaaggga gtcgatgtcc agggcacgct ttccaaaatt tttaagctgg gaggaagaga |
| 541 | tagtcgtact ggatcaccca tggctagacg ctgaaaaccc acctggttcc ggaatcctgt |
| 601 | cctcagcttc ttaatataac tgccttaaaa ctttaatccc acttgcccct gttacctaat |
| 661 | tagagcagat gacccctccc ctaatgcctg cggagttgtg cacgtagtag ggtcaggcca |
| 721 | cggcagccta ccggcaattt ccggccaaca gttaaatgag aacatgaaaa cagaaaacgg |
| 781 | ttaaaactgt ccctttctgt gtgaagatca cgttccttcc cccgcaatgt gcccccagac |
| 841 | gcacgtgggt cttcaggggg ccaggtgcac agacgtccct ccacgttcac ccctccaccc |
| 901 | ttggactttc ttttcgccgt ggctgcggca cccttgcgct tttgctggtc actgccatgg |
| 961 | aggcacacag ctgcagagac agagaggacg tgggcggcag agaggactgt taacatccaa |
| 1021 | gcttcctttg tttttttttc ctgtccttct ctcacctcct aaagtagact tcatttttcc |
| 1081 | taacaggatt agacagtcaa agagtgactt actacatgtg ggagcttttg gtatgtaaca |
| 1141 | tgcgggctgg gcagctgtta gagtccaacg tggggcagca cagagagggg gccacctccc |
| 1201 | caggccgtgg ctacccacac accccaatta gctgaattcg cgtatggcag agggaggaaa |
| 1261 | aggaggcaaa cgtgggctgg gcaatggcct cacataggaa acagggtctt cctggagatt |
| 1321 | tggtgatgga gatgtcaagc aggtggcctc tggacgtcac cgttgccctg catggtggcc |
| 1381 | ccagagcagc ctctatgaac aacctcgttt ccaaaccaca gcccacagcc ggagagtcca |
| 1441 | aaaagacttg cgcactcaga gcagaagggt aggagtcctc tagacaacct cgcaaccgcg |
| 1501 | ccagtcgccc atagacactg actgtgaccg ggcgtactgg cagcggcagt gcacagtggc |
| 1561 | cagcactaac cctccctgag aagataacca gctcattcac ttcctcccaa aagacgcgtg |
| 1621 | gtagcgagta ggcacaggcg tgcacctgct cccgaattac tcaccgagac acacgggctg |
| 1681 | agcagacggc cccgtggatg gagacaaaga gctcttctga ccatatcctt cttaacaccc |
| 1741 | gctggcatct cctttcgcgc ctccctccct aacctactga cccacctttt gattttagcg |
| 1801 | cacctgtgat tgataggcct tccaaagagt cccacgctgg catcaccctc cccgaggacg |
| 1861 | gagatgagga gtagtcagcg tgatgccaaa acgcgtcttc ttaatccaat tctaattctg |
| 1921 | aatgtttcgt gtgggcttaa taccatgtct attaatatat agcctcgatg atgagagagt |
| 1981 | tacaaagaac aaaactccag acacaaacct ccaaattttt cagcagaagc actctgcgtc |
| 2041 | gctgagctga ggtcggctct gcgatccata cgtggccgca cccacacagc acgtgctgtg |
| 2101 | acgatggctg aacggaaagt gtacactgtt cctgaatatt gaaataaaac aataaacttt |
| 2161 | taatggtaaa aaaaaaaaaa aaaaaaaaa |

Embodiments of the present disclosure use MBPs, which will be understood to include genetic variants (mutations) and fragments thereof, to disrupt aggregations of fibrillizing peptides.

As used herein, a “fragment” of a protein encompasses any sequence of amino acids that (1) matches a sequence of amino acids within a given protein and (2) is shorter than the protein by at least one amino acid residue. The term “match,” as used herein, encompasses any sequence that does not, in a sequence that otherwise would provide an“interfering fragment” (i.e., a fragment that interferes with a fibrillization of a fibrillizing peptide), contain an amino acid that inhibits the interference. In other words, a “match” need not be an identical match. Homologous sequences that do not erase function in the fragment are treated as matches. Such fragments may be referred to herein, interchangeably, as “interfering fragments” or “functional fragments.” A fragment is functional if it inhibits (i.e., “interferes with” or reduces the rate at which an action proceeds) the formation of (1) complexes of fibrillizing peptide bound to the fragment, (2) peptide oligomers, or (3) peptide fibrils. In some embodiments, the fragment forms a complex by binding with Aβ. In some embodiments the fragment comprises the motif KRGX₁X₂X₃X₄X₅X₆HP (SEQ ID NO: 1), wherein X₁-X₆ are amino acids selected from the group consisting of G, P, A, V, L, I, M, C, F, Y, W, H, K, R, Q, N, E, D, S and T.

Other embodiments use MBPs to prevent aggregations from forming. The term “disrupt,” however, as used herein, is not intended to exclude “formation” or vice versa, since embodiments of the disclosure are not limited by any theory as to precisely how aggregates form or what dynamics may then govern their maintenance. In other embodiments, the disclosure employs MBPs to treat amyloidoses, particularly amyloidoses associated with anomalies in the metabolism of Aβ-42 and/or Aβ-40 mutants. An “anomaly in metabolism,” as used herein, encompasses any production or accumulation, in any form, of Aβ-42 peptide and/or Aβ-40 mutant peptide in a cell or tissue of a subject, which production or accumulation a person of skill in the healing arts deems to be pathogenic or pathological. The term is intended to include any condition deemed to indicate that the subject has a propensity for such an anomaly.

“Fibrillization,” as used herein, refers to a process that drives certain peptides to aggregate, ultimately to form complex, often heterogenous, and typically insoluble structures. Peptides susceptible to this process, often called “amyloid peptides,” are also referred to herein as “fibrillizing peptides.”

In some embodiments, the disclosure provides a MBP that digests fibril precursors, thus depriving the fibrillization process of its substrate. In some embodiments, a MBP changes fibril precursors into forms that do not or cannot assemble or fibrillize. In some embodiments, a MBP interferes with fibrillization by disrupting an insoluble fibrillar product of the fibrillization process. In some embodiments, the invention provides a MBP or fragment thereof that interferes with fibrillization, such that fibrillization is slowed or prevented, thus preventing an amyloid disease or at least inhibiting the progression of the disease or reducing the severity of its symptoms.

In one embodiment of the present disclosure, an abnormal condition can be prevented or treated by using an MBP or a fragment thereof linked to a therapeutic agent to deliver the agent to a target, the target being defined as a target site for the MBP or a binding site for the MBP fragment. The “therapeutic agent” may be any agent that confers any therapeutic effect on a subject. As a non-limiting example, MBP or a fragment of MBP may be employed, by loading onto an O-GNR, to deliver to sites of accumulation in the brain of a subject. Also, and again without limitation, a cell, a viral particle, a macromolecule or a non-biological particle may be conjugated with the MBP or fragment thereof. An O-GNR loaded with MBP or a fragment thereof is referred to herein as a “targeting moiety.”

MBP or fragments thereof may be administered in relevant embodiments of the disclosure by a variety of routes, including but not limited to topical, oral, nasal, enteral, intravenous, intraarterial, intramuscular, intracardiac, subcutaneous, intradermal, intrathecal, epidural, intracranial, cerebro-ventricular, brain parenchymal, intraperitoneal, intravesical, inhalational, and intraocular. In certain embodiments of the disclosure are methods wherein stem cells are stably transfected ex vivo with a nucleic acid sequence that encodes an MBP or a fragment thereof and transplanted to or near a site where a fibrillizing peptide is expressed or tends to accumulate in a subject.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A method for delivering a drug to a mammal's cell, the method comprising: administering a carbon nanomaterial to the mammal, wherein the carbon nanomaterial comprises the drug loaded onto the carbon nanomaterial, and wherein the carbon nanomaterial is configured to enter into the cell.

2. The method of claim 1, wherein the carbon nanomaterial is graphene.

3. The method of claim 1, wherein the carbon nanomaterial is graphene nanoribbons.

4. The method of claim 1, wherein the carbon nanomaterial is oxidized graphene nanoribbons.

5. The method of claim 1, wherein the carbon nanomaterial is combined with at least one of 1, 2-distearoyl-sn-glycero-3-phosphoethanolamine-N-[amino (polyethylene glycol)), lucanthone, doxorubicin, paclitaxel, camptothecin, tamoxifen and ceramide.

6. The method of claim 1, wherein the cell is a glioblastoma cell.

7. The method of claim 1, wherein the carbon nonmaterial is coated with a polymer.

8. A carbon nanomaterial comprising:
a carbon nanomaterial; and
a drug loaded onto the carbon nanomaterial.

9. The nanomaterial of claim 8 wherein the nanomaterial is oxidized graphene nanoribbons.

10. The nanomaterial of claim 8, further comprising a polymer that is configured to coat the carbon nanomaterial.

11. The nonmaterial of claim 10, wherein the polymer is selected from the group consisting of 1, 2-distearoyl-sn-glycero-3-phosphoethanolamine-N-[amino (polyethylene glycol)), dextran and combinations thereof.

12. The nanomaterial of claim 8, wherein the drug is selected from the group consisting of lucanthone, doxorubicin, paclitaxel, camptothecin, tamoxifen, ceramide and combinations thereof.

13. A method for delivering a nucleic acid to a mammal's cell, the method comprising:
administering a carbon nanomaterial to the mammal, wherein the carbon nanomaterial further comprises the nucleic acid, and wherein the carbon nonmaterial is configured to enter into the cell.

14. The method of claim 11, wherein the carbon nanomaterial is graphene.

15. The method of claim 11, wherein the carbon nanomaterial is graphene nanoribbons.

16. The method of claim 11, wherein the carbon nanomaterial is oxidized graphene nanoribbons.

17. The method of claim 11, wherein the nucleic acid is at least one of a plasmid, DNA molecule and siRNA molecule.

18. The method of claim 11, wherein the cell is a cervical cancer cell.

19. The method of claim 11, wherein the carbon nanomaterial further comprises a polymer that is configured to coat the carbon nanomaterial.

20. A carbon nanomaterial comprising:
a carbon nanomaterial; and
a nucleic acid loaded onto the coated carbon nanomaterial.

21. The nanomaterial of claim 18, wherein the nanomaterial is oxidized graphene nanoribbons.

22. The nanomaterial of claim 18, wherein the nucleic acid is at least one of a plasmid, DNA molecule and siRNA molecule.

23. A method for delivering a substance to a mammal's cell, the method comprising: administering a carbon nanomaterial to the mammal, wherein the carbon nanomaterial comprises the substance, the substance selected from the group consisting of one or more proteins, one or more nucleic acids and one or more proteins and one or more nucleic acids, and wherein the carbon nanomaterial is configured to enter into the cell.

24. The method of claim 23, wherein the mammal's cell is a brain cell.

25. The method of claim 23, wherein the protein is myelin basic protein (MBP) or a fragment thereof.

26. The method of claim 23, wherein the carbon nanomaterial is oxidized graphene nanoribbons.

27. A carbon nanomaterial comprising:
a carbon nanomaterial; and
a substance loaded onto the carbon nanomaterial, the substance selected from the group consisting of one or more proteins, one or more nucleic acids and one or more proteins and one or more nucleic acids.

28. The nanomaterial of claim 27 wherein the nanomaterial is oxidized graphene nanoribbons.

29. The nanomaterial of claim 27, wherein the protein is myelin basic protein (MBP) or a fragment thereof.
